(19) United States
(12) Patent Application Publication
(10) Pub. No.: US 2025/0057128 A1
(43) Pub. Date: Feb. 20, 2025

(54) HUMANIZED MOUSE MODELS

(71) Applicant: The Jackson Laboratory, Bar Harbor, ME (US)

(72) Inventor: James Keck, Bar Harbor, ME (US)

(73) Assignee: The Jackson Laboratory, Bar Harbor, ME (US)

(21) Appl. No.: 18/723,361

(22) PCT Filed: Dec. 21, 2022
(86) PCT No.: PCT/US2022/053588

§ 371 Date: Jun. 21, 2024

Related U.S. Application Data
(60) Provisional application No. 63/292,757, filed on Dec. 22, 2021.

(51) Int. Cl. A01K 67/0278; C07K 14/535; C07K 14/54; C12N 15/85

(52) U.S. Cl. CPC A01K 67/0278; C07K 14/535; C07K 14/5443; C12N 15/85; A01K 2207/15; A01K 2227/105; A01K 2267/0331

(57) ABSTRACT

The human immune system is complex, and its related diseases are similarly complicated. Therefore, human diseases, such as cancers, are often difficult to characterize and treat effectively. Immunodeficient transgenic mice for use as humanized mouse models are provided. The humanized mouse models may be further engrafted with diseased tissues or cells (e.g., human cancer cells). Methods of using the humanized mouse models to assess disease progression, immune response, and efficacy of proposed therapeutic agents are also provided herein.

### BACKGROUND

The human immune system is complex, and its related diseases are similarly complicated. Therefore, human diseases, such as cancers, are often difficult to characterize and treat effectively. There is a continuing need for animal models that allow for isolation of aspects of the immune response, providing methods and compositions useful, for example, for identifying effective medical and pharmaceutical therapies.

### SUMMARY

The present disclosure provides, in some aspects, an immunodeficient transgenic mouse comprising a transgene encoding human interleukin-3 (IL-3), a transgene encoding human granulocyte/macrophage-colony stimulating factor 2 (GM-CSF), a transgene encoding human stem cell factor (SCF), and a transgene encoding human IL-15. As described herein, the immunodeficient transgenic mouse represents an improved humanized mouse model due at least in part to its physiological properties, which more closely mimic those of humans, relative to previous humanized mouse models. Humanization of the immunodeficient transgenic mouse provided herein enables disease modeling and assessment of therapeutic agents (e.g., candidate therapeutic agents). Unexpectedly, the data provided herein shows that exposing the immunodeficient mice to myeloablative therapy, such as irradiation, enhances immunoglobulin production, separately from humanization. Interestingly, different doses of irradiation lead to different outcomes. Mice exposed to 50 cGy of irradiation, for example, produced the highest human immunoglobulin levels of any dose tested.

Some aspects provide a method of producing a mouse model of human humoral and cellular immunity, the method comprising: (a) providing an immunodeficient mouse comprising a transgene encoding human interleukin-3 (IL-3), a transgene encoding human granulocyte/macrophage-colony stimulating factor 2 (GM-CSF), a transgene encoding human stem cell factor (SCF), and a transgene encoding human IL-15; (b) administering a myeloablative treatment to the immunodeficient mouse; and (c) administering human peripheral blood mononuclear cells (hPBMCs) to the immunodeficient mouse.

In some embodiments, the myeloablative treatment is irradiation.

In some embodiments, the irradiation is gamma irradiation (cGy). In some embodiments, the myeloablative treatment is ≤150 cGy. In some embodiments, the myeloablative treatment is ≤100 cGy. In some embodiments, the myeloablative treatment is about 50 to about 100 cGy. In some embodiments, the myeloablative treatment is 40-60 cGy. In some embodiments, the myeloablative treatment is about 50 cGy.

In some embodiments, fewer than 1×10⁷ of the hPBMCs are administered to the immunodeficient mouse. In some embodiments, 0.5×10⁶-5×10⁶ of the hPBMCs are administered to the immunodeficient mouse.

In some embodiments, the administering of the hPBMCs is within 3 days of the administering of the myeloablative treatment. In some embodiments, the administering of the hPBMCs is on the same day as the administering of the myeloablative treatment. In some embodiments, the administering of the hPBMCs is within 6 hours of the administering of the myeloablative treatment.

In some embodiments, the method further comprises administering to the immunodeficient mouse human diseased cells.

In some embodiments, the method further comprises administering to the immunodeficient mouse a human therapeutic or prophylactic agent (or a candidate human therapeutic agent).

In some embodiments, the human therapeutic agent (or a candidate human therapeutic agent) is selected from human immunomodulatory agents. In some embodiments, the human therapeutic agents (or a candidate human therapeutic agents) are selected from monoclonal antibodies. In some embodiments, the human immunomodulatory agents are selected from cellular therapies, optionally T cell therapies.

In some embodiments, the method further comprises administering to the immunodeficient mouse a human therapeutic or prophylactic agent is a vaccine.

In some embodiments, the vaccine is a protein antigen or a nucleic acid encoding a protein antigen, optionally a cancer antigen or a pathogenic antigen.

In some embodiments, the administering of the human therapeutic agent (or a candidate human therapeutic agent) is within 30 days of administering the myeloablative treatment. In some embodiments, the administering of the human therapeutic agent (or a candidate human therapeutic agent) is within 28 days of administering the myeloablative treatment. In some embodiments, the administering of the human therapeutic agent (or a candidate human therapeutic agent) is within 21 days of administering the myeloablative treatment. In some embodiments, the administering of the human therapeutic agent (or a candidate human therapeutic agent) is within 14 days of administering the myeloablative treatment.

In some embodiments, the method further comprises assaying a sample from the mouse for one or more human cytokines. The sample may be, for example, a blood sample.

In some embodiments, the one or more human cytokines are selected from interferon gamma (IFN-γ), interleukin (IL)-2, IL-4, IL-6, IL-10, and tumor necrosis factor alpha (TNFα).

In some embodiments, the method further comprises assaying a sample from the mouse for one or more human immunoglobulins. The sample may be, for example, a blood sample.

In some embodiments, the one or more human immunoglobulins are selected from IgM, IgA, IgG, optionally IgG1, IgG2, IgG3, and IgG4.

In some embodiments, the method further comprises assaying a sample, optionally a blood sample, from the mouse for one or more anti-drug antibodies.

Some aspects provide a mouse model of human humoral and cellular immunity produced by the method of any one of the preceding aspects and/or embodiments.

Other aspects provide an immunodeficient mouse comprising a transgene encoding human interleukin-3 (IL-3), a transgene encoding human granulocyte/macrophage-colony stimulating factor 2 (GM-CSF), a transgene encoding human stem cell factor (SCF), and a transgene encoding human IL-15.

In some embodiments, the immunodeficient mouse has a non-obese diabetic genetic background.

In some embodiments, the immunodeficient mouse has a severe combined immune deficiency mutation (Prkdc^{scid}).

In some embodiments, the immunodeficient mouse has a null allele of the IL2 receptor common gamma chain (IL2rg^{null}).

In some embodiments, the immunodeficient mouse has a NOD-scid IL2Rgamma^{null} genetic background.

In some embodiments, the immunodeficient mouse has be exposed to myeloablative treatment. In some embodiments, the myeloablative treatment is irradiation.

In some embodiments, cells of the immunodeficient mouse produce human IgM, IgG1, IgG2, IgG3, and IgG4.

In some embodiments, cells of the immunodeficient mouse produce 6000-16000 μl/mL of human IgG. In some embodiments, cells of the immunodeficient mouse produce 1000-2000 μl/mL of human IgM. In some embodiments, cells of the immunodeficient mouse produce 1500-2500 μl/mL of human IgG1. In some embodiments, cells of the immunodeficient mouse produce 500-1500 μl/mL of human IgG2. In some embodiments, cells of the immunodeficient mouse produce 150-205 μl/mL of human IgG3. In some embodiments, cells of the immunodeficient mouse produce 50-1000 μl/mL of human IgG4.

In some embodiments, the immunodeficient mouse produces human CD3⁺, CD4⁺, CD8⁺ and CD19⁺ cells.

### BRIEF DESCRIPTION OF THE DRAWINGS

FIGS. 1A-1F show comparisons of human CD45+ engraftment in NSG-SGM3 mice and NSG-SGM3×IL15 mice engrafted with CD34+ hemopoietic stem cells (HSCs) from three different donors. FIGS. 1A-1C demonstrate the frequency of CD45+ cells from three different donors, while FIGS. 1D-1F demonstrate the corresponding CD45+ cells/microliter from the three donors.

FIGS. 2A-2F show comparisons of human natural killer (NK) cells, T cells, B cells, and myeloid cells in NSG-SGM3 mice and NSG-SGM3×IL15 mice engrafted with CD34+ HSCs from three different donors over time. FIGS. 2A-2C demonstrate the percentage of the different human immune cells in NSG-SGM3 mice, and FIGS. 2D-2F demonstrate the percentage of the different human immune cells in NSG-SGM3×IL15 mice. The arrows are pointing to the NK cell data.

FIGS. 3A-3F show comparisons of human NK cells in NSG-SGM3 mice and NSG-SGM3×IL15 mice engrafted with CD34+ HSCs from three different donors over time. FIGS. 3A-3C show NK cells as a percentage of CD45⁺ cells (cell frequency) and FIGS. 3D-3F show NK cells/microliter.

FIGS. 4A-4F show comparisons of tumor growth kinetics in NSG® mice, NSG-SGM3 mice and NSG-SGM3×IL15 mice engrafted with CD34+ HSCs and orthotopically implanted with a human cell line-derived xenograft (CDX; MDA-MB-231, FIGS. 4A and 4D) or subcutaneously implanted with a patient-derived xenograft (PDX) over time. The two patient-derived xenografts (PDXs) used were: PS4050 (FIGS. 4B and 4E) or LG1306 (FIGS. 4C and 4F). FIGS. 4A-4C show the mean tumor volume of the three xenograft models, and FIGS. 4D-4F show the individual tumor size over time.

FIGS. 5A-5B show the frequency of human regulatory T (Treg) cells in the blood (FIG. 5A) and PD-1 expression on CD4+ T cells and on CD8+ T cells in the spleens (FIG. 5B) of NSG-SGM3 mice and NSG-SGM3×IL15 mice engrafted with CD34+ HSCs.

FIGS. 6A-6C show comparisons of leukocytes (FIG. 6A), human myeloid cells (FIG. 6B), and human NK cells (FIG. 6C) in NSG-SGM3 mice and NSG-SGM3×IL15 mice engrafted with 5×10⁶ human peripheral blood mononuclear cells (PBMCs) per mouse.

FIGS. 7A-7C show comparisons of activated T cells in the spleens of NSG-SGM3 mice and NSG-SGM3×IL15 mice engrafted with 5×10⁶ human PBMCs per mouse.

FIGS. 8A-8C show comparisons of human CD45+ cells (FIG. 8A), human myeloid cells (FIG. 8B), and human NK cells (FIG. 8C) in NSG-SGM3 mice and NSG-SGM3×IL15 mice engrafted with 1×10⁶ or 5×10⁶ human PBMCs per mouse, with two different doses of irradiation (150 and 175 cGy).

FIGS. 9A-9C show comparisons of TNF (FIG. 9A), IL-6 (FIG. 9B), and IL-2 (FIG. 9C) levels in NSG-SGM3 mice and NSG-SGM3×IL15 mice engrafted with 5×10⁶ human PBMCs per mouse.

FIGS. 10A-10C show comparisons of IFN-γ (FIG. 10A), TNF (FIG. 10B), and IL-6 (FIG. 10C) levels in NSG-SGM3 mice and NSG-SGM3×IL15 mice engrafted with 1×10⁶ or 2×10⁶ human PBMCs per mouse, with two different doses of irradiation (150 and 175 cGy).

FIGS. 11A-11B show comparisons of humanization in NSG-SGM3×IL15 mice, with or without irradiation, engrafted with PBMCs from a single donor. FIG. 11A demonstrates human CD45+ cells as cells/μL at 7, 14 and 21 days post-engraftment. FIG. 11B demonstrates human CD45+ cells as a percentage of total live cells at 7, 14 and 21 days post-engraftment.

FIGS. 12A-12B show comparisons of humanization in NSG-SGM3×IL15 mice, with or without irradiation, engrafted with PBMCs from two different donors. FIG. 12A demonstrates human CD45+ cells as a percentage of total live cells at 14 and 21 days post-engraftment. FIG. 12B demonstrates human CD45+ cells as cells/μL at 14 and 21 days post-engraftment.

FIG. 13 shows a comparison of circulating human IgG levels in NSG-SGM3×IL15 mice, with or without irradiation, at 14 and 21 days post-engraftment with PBMCs from three different donors.

FIGS. 14A-14B show comparisons of human Ig isotypes in serum of NSG-SGM3×IL15 mice, with or without irradiation, at 14 (FIG. 14A) and 21 days (FIG. 14B) post-engraftment with PBMCs from a single donor.

FIG. 15A-15B show comparisons of human immune cell population in NSG-SGM3×IL15 mice, with or without irradiation, at 14 (FIG. 15A) and 21 days (FIG. 15B) post-engraftment with PBMCs from a single donor.

FIG. 16A-16B show comparisons of humanization (FIG. 16A) and circulating human immunoglobulin G (hIgG) levels (FIG. 16B) in PBMC-engrafted NSG-SGM3×IL15 mice at various dosages of irradiation.

### DETAILED DESCRIPTION

The present disclosure provides, in some embodiments, an immunodeficient mouse model expressing human IL-3, human granulocyte/macrophage-colony stimulating factor 2 GM-CSF, human SCF, and a human IL-15. In some embodiments, the mouse is produced by crossing the NSG-SGM3 mouse with the NSG-IL-15 mouse. The mouse, in some embodiments, is humanized with CD34+ hemopoietic stem cells (HSCs) or peripheral blood mononuclear cells (PBMCs), and unexpectedly has a more physiological and expanded humanized immune profile relative to either of the two parental strains. For example, as described herein, when the mouse model is humanized with PBMCs, higher levels of human immune cells (e.g., CD45+ cells) than either of the parent strains are produced. In this way, surprisingly low numbers of patient PBMCs (e.g., as few as 1×10⁶ patient PBMCs) may be used to humanize the mouse. This is important, as patient PBMCs are often present in very low numbers, especially in the early phases of disease. The human immune cell population in the mouse model provided herein also has higher levels of activated T cells and higher levels of natural killer (NK) cells in the blood, relative to the parental strains. Similarly, human physiological levels of T regulatory cells and PD-1+ T cells are produced in the mice, thereby increasing sensitivity for screening immunomodulatory agents, such as checkpoint inhibitor agents. In some embodiments, the mouse model comprises significantly more human T cells, human B cells, and/or human NK cells in peripheral blood, relative to a humanized NSG-SGM3 mouse.

Methods of producing the mouse models, humanizing the mouse models, and of using the mouse models, for example, to assess disease progression (e.g., cancer and/or other diseases of the human immune system) and therapeutic agents (e.g., candidate therapeutic agents) are also provided herein. The models provided herein, in some embodiments, are further engrafted with cells, such as mammalian (e.g., human) cells, for example, human diseased cells, such as human cancer cells. In some embodiments, the human cells are from a patient-derived xenograft (PDX) or a human cell line (e.g., a human cancer cell line). Diseased cells and tissues are associated with unique genetic profiles that are used herein to produce mouse models that can be utilized, for example, to explore the genetic components that may underly certain diseases (e.g., cancer, autoimmune diseases, and other inflammatory diseases). By replicating the human immune system, these models may also be used in some embodiments to evaluate toxicity (e.g., cytokine release syndrome) and other side effects of certain therapeutics aimed at preventing or treating disease.

An animal model may be, but is not limited to, a non-human mammal, a rodent (e.g., a mouse, a rat, or a hamster), or a livestock animal (e.g., a pig, a cow, a chicken, or a goat) model. In some embodiments, the animal is a rodent. In some embodiments, the animal is a mouse.

Herein, for simplicity, reference is made to “mouse” and “mouse models” (e.g., surrogates for human conditions). It should be understood that these terms, unless otherwise stated, may be used interchangeably throughout the specification to encompass “rodent” and “rodent models,” including mouse, rat and other rodent species.

It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The registry is maintained at the Institute for Laboratory Animal Research (ILAR) at the National Academy of Sciences, Washington, D.C. Lab Codes may be obtained electronically at ILAR's web site (nas.edu/cls/ilarhome.nsf). See also Davisson M T, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

A mouse model of disease may be modified to enable the assessment of a disease. Any system (e.g., immune, respiratory, nervous, or circulatory), organ (e.g., blood, heart, blood vessels, spleen, thymus, lymph nodes, or lungs), tissue (e.g., epithelial, connective, muscle, and nervous), or cell type (e.g., lymphocytes or macrophages) may be modified, either independently or in combination, to enable studying disease in the models provided herein.

Three conventional methods used for the production of transgenic mice include DNA microinjection (Gordon and Ruddle, Science 1981: 214: 1244-124, incorporated herein by reference), embryonic stem cell-mediated gene transfer (Gossler et al., Proc. Natl. Acad. Sci. 1986, 83: 9065-9069, incorporated herein by reference) and retrovirus-mediated gene transfer (Jaenisch, Proc. Natl. Acad. Sci. 1976, 73: 1260-1264, incorporated herein by reference), any of which may be used as provided herein. Genomic editing methods using, for example, clustered regularly interspace palindromic repeats (CRISPR/Cas) nucleases, transcription activator-like effector nucleases (TALENs), or zinc finger nucleases (ZFNs) are described elsewhere herein.

Following delivery of nucleic acids to a fertilized embryo (e.g., a single-cell embryo (e.g., a zygote) or a multi-cell embryo (e.g., a developmental stage following a zygote, such as a blastocyst), the fertilized embryo is transferred to a pseudopregnant female, which subsequently gives birth to offspring. The presence or absence of a nucleic acid encoding human FcRn and/or a chimeric IgG antibody may be confirmed, for example, using any number of genotyping methods (e.g., sequencing and/or genomic PCR).

New mouse models can also be created by breeding parental lines, as described in the Examples herein. With the variety of available mutant, knock-out, knock-in, transgenic, Cre-lox, Tet-inducible system, and other mouse strains, multiple mutations and transgenes may be combined to generate new mouse models. Multiple mouse strains may be bred together to generate double, triple, or even quadruple and higher multiple mutant/transgenic mice.

In some embodiments, parental mice are bred to produce F1 mice. A parental mouse may be, for example, homozygous, heterozygous, hemizygous, or homozygous null at a particular allele. Homozygous describes a genotype of two identical alleles at a given locus, heterozygous describes a genotype of two different alleles at a locus, hemizygous describes a genotype consisting of only a single copy of a particular gene in an otherwise diploid organism, and homozygous null refers to an otherwise-diploid organism in which both copies of the gene are missing.

### Immunodeficient Mouse Models

Provided herein, in some embodiments, are immunodeficient mouse models comprising a transgene encoding human interleukin-3 (IL-3), a transgene encoding human granulocyte/macrophage-colony stimulating factor 2 (GM-CSF), a transgene encoding human stem cell factor (SCF), and a transgene encoding human IL-15.

As is known in the art, immunodeficient mice have impaired or disrupted immune systems, such as specific deficiencies in MHC class I, II or both, B cell or T cell defects, or defects in both, natural killer cell defects, myeloid defects (e.g., defects in granulocytes and/or monocytes), as well as immunodeficiency due to knockdown of genes for cytokines, cytokine receptors, TLR receptors and a variety of transducers and transcription factors of signaling pathways. Immunodeficiency mouse models include the single-gene mutation models such as nude-mice (nu) strains and the severe combined immunodeficiency (scid) strains, non-obese diabetic (NOD) strain, RAG (recombination activating gene) strains with targeted gene deletion and a variety of hybrids originated by crossing doubly and triple mutation mice strains with additional defects in innate and adaptive immunity.

Non-limiting examples of spontaneous and transgenic immunodeficient mouse models include the following mouse strains:
Nude (nu) [Flanagan S P. Genet Res 1966; 8: 295-309; and Nehls M et al. Nature 1994; 372: 103-7];
Scid (scid) [Bosma G C et al. Nature 1983; 301:527-30; Mosier D E et al. Nature 1988; 335: 256-9; and Greiner D L et al. Stem Cells 1998; 16: 166-77];
NOD [Kikutani H et al. Adv Immunol 1992; 51: 285-322; and Anderson M S et al. Ann Rev Immunol 2005; 23: 447-85];
RAG1 and RAG2 (rag) [Mombaerts P et al. Cell 1992; 68: 869-77; Shinkai U et al. Cell 1992; 68: 855-67];
NOD-scid [Greiner D L et al. 1998; Shultz L D et al. J Immunol 1995; 154: 180-91; Melkus M W et al. Nature Med 2006; 12: 1316-22; and Denton P W et al. PLoS Med 2008; 4(12): e357];
IL2rgnull [DiSanto J P et al. Proc Natl Acad Sci USA 1995; 92: 377-81];
B2mnull [Christianson S W et al. J Immunol 1997; 158: 3578-86];
NOD-scid IL2rynull [Shultz L D et al. Nat Rev Immunol 2007; 7: 118-30; Ito M et al. Blood 2002; 100: 3175-82; Ishikawa I et al. Blood 2005; 106: 1565-73; and Macchiarini F et al. J Exp Med 2005; 202: 1307-11];
NOD-scid B2mnull [Shultz et al. 2007; Shultz L D et al. Transplantation 2003; 76: 1036-42; Islas-Ohlmayer M A et al. J Virol 2004; 78:13891-900; and Macchiarini et al. 2005];
HLA transgenic mice [Grusby M J et al. Proc Natl Acad Sci USA 1993; 90(9): 3913-7; and Roy C J et al. Infect Immun 2005; 73(4): 2452-60]. See, e.g., Belizario J E The Open Immunology Journal, 2009; 2:79-85;
NOG mice (NOD.cg-Prkdc^{scid}Il2rg^{tm1Sug}) [Shultz L D et al. Nat Rev Immunol 2007; 7: 118-30]; and
BRG mice (BALB/c;129S4-Rag2^{tm1.1Flv}) [Song J et al. Cell Host Microbe 2010; 8(4): 369-76; Goldman J P et al. Br J Haematol. 1998; 103: 335-342].

Provided herein, in some embodiments, are immunodeficient mouse models having the non-obese diabetic (NOD) mouse genotype. The NOD mouse (e.g., Jackson Labs Stock #001976, NOD-Shi^{LtJ}) is a polygenic mouse model of autoimmune (e.g., Type 1) diabetes, characterized by hyperglycemia and insulitis, a leukocytic infiltration of the pancreatic islet cells. The NOD mice are hypoinsulinemic and hyperglucagonemic, indicating a selective destruction of pancreatic islet beta cells. The major component of diabetes susceptibility in NOD mice is the unique MHC haplotype. NOD mice also exhibit multiple aberrant immunophenotypes including defective antigen presenting cell immunoregulatory functions, defects in the regulation of the T lymphocyte repertoire, defective NK cell function, defective cytokine production from macrophages (Fan et al., 2004) and impaired wound healing. They also lack hemolytic complement, C5. NOD mice also are severely hard-of-hearing. A variety of mutations causing immunodeficiencies, targeted mutations in cytokine genes, as well as transgenes affecting immune functions, have been backcrossed into the NOD inbred strain background.

In some aspects of the present disclosure, an immunodeficient mouse provided herein based on the NOD background may have a genotype selected from NOD-Cg.-Prkdc^{scid}IL2rg^{tm1wJl}/SzJ (NSG®), a NOD.Cg-Rag1^{tm1Mom}Il2rg^{tm1Wjl}/SzJ (NRG), and NOD.Cg-Prkdc^{scid}IL2rg^{tm1Sug}/ShiJic (NOG). Other immunodeficient mouse strains are contemplated herein.

In some embodiments, an immunodeficient mouse model based on the NOD background has an NOD-Cg.-Prkdc^{scid}IL2rg^{tm1wJl}/SzJ (NSG™) genetic background. The NSG™ mouse (e.g., Jackson Labs Stock No.: #005557) is an immunodeficient mouse that lacks mature T cells, B cells, and NK cells, is deficient in multiple cytokine signaling pathways, and has many defects in innate immune immunity (see, e.g., Shultz, Ishikawa, & Greiner, 2007; Shultz et al., 2005; and Shultz et al., 1995, each of which is incorporated herein by reference). The NSG™ mouse, derived from the NOD mouse strain NOD/ShiLtJ (see, e.g., Makino et al., 1980, which is incorporated herein by reference), includes the Prkdc^{scid} mutation (also referred to as the “severe combined immunodeficiency” mutation or the “scid” mutation) and the Il2rg^{tm1wJl} targeted mutation. The Prkdc^{scid} mutation is a loss-of-function (null) mutation in the mouse homolog of the human PRKDC gene—this mutation essentially eliminates adaptive immunity (see, e.g., (Blunt et al., 1995; Greiner, Hesselton, & Shultz, 1998), each of which is incorporated herein by reference). The Il2rg^{tm1wJl} mutation is a null mutation in the gene encoding the interleukin 2 receptor gamma chain (IL2R7, homologous to IL2RG in humans), which blocks NK cell differentiation, thereby removing an obstacle that prevents the efficient engraftment of primary human cells (Cao et al., 1995; Greiner et al., 1998; and Shultz et al., 2005, each of which is incorporated herein by reference). In some embodiments, the mouse model comprises an NSG-SGM3 mouse crossed with an NSG-IL-15 mouse.

In some embodiments, an immunodeficient mouse model has an NRG genotype. The NRG mouse (e.g., Jackson Labs Stock #007799) is extremely immunodeficient. This mouse comprises two mutations on the NOD/ShiLtJ genetic background; a targeted knockout mutation in recombination activating gene 1 (Rag1) and a complete null allele of the IL2 receptor common gamma chain (IL2rg^{null}). The Rag1^{null} mutation renders the mice B and T cell deficient and the IL2rg^{null} mutation prevents cytokine signaling through multiple receptors, leading to a deficiency in functional NK cells. The extreme immunodeficiency of NRG allows the mice to be humanized by engraftment of human CD34⁺ hematopoietic stem cells (HSC) and patient derived xenografts (PDXs) at high efficiency. The immunodeficient NRG mice are more resistant to irradiation and genotoxic drugs than mice with a scid mutation in the DNA repair enzyme Prkdc.

In some embodiments, an immunodeficient mouse model is an NOG mouse. The NOG mouse (Ito M et al., Blood 2002) is an extremely severe combined immunodeficient (scid) mouse established by combining the NOD/scid mouse and the IL-2 receptor-γ chain knockout (IL2rγKO) mouse (Ohbo K. et al., Blood 1996). The NOG mouse lacks T and B cells, lacks natural killer (NK) cells, exhibits reduced dendritic cell function and reduced macrophage function, and lacks complement activity.

In some embodiments, an immunodeficient mouse model has an NCG genotype. The NCG mouse (e.g., Charles River Stock #572) was created by sequential CRISPR/Cas9 editing of the Prkdc and Il2rg loci in the NOD/Nju mouse, generating a mouse coisogenic to the NOD/Nju. The NOD/Nju carries a mutation in the Sirpa (SIRPα) gene that allows for engrafting of foreign hematopoietic stem cells. The Prkdc knockout generates a SCID-like phenotype lacking proper T-cell and B-cell formation. The knockout of the Il2rg gene further exacerbates the SCID-like phenotype while additionally resulting in a decrease of NK cell production.

Provided herein, in some embodiments, are immunodeficient mouse models that are deficient in (e.g., lacks) MHC Class I, MHC Class II, or MHC Class I and MHC Class II. A mouse that is deficient in MHC Class I and/or MHC Class II does not express the same level of MHC Class I proteins (e.g., α-microglobulin and β2-microglobulin (B2M)) and/or MHC Class II proteins (e.g., α chain and β chain) or does not have the same level of MHC Class I and/or MHC Class II protein activity as a non-immunodeficient (e.g., MHC Class I/II wild-type) mouse. In some embodiments, the expression or activity of MHC Class I and/or MHC Class II proteins is reduced (e.g., by at least 30%, at least 40%, at least 50%, at least 60%, at least 70%, at least 80%, at least 90% or more), relative to a non-immunodeficient mouse. Immunodeficient mice that are deficient in MHC Class I, MHC Class II, and MHC Class I and MHC Class II are described in International Publication No. WO 2018/209344, the content of which is incorporated by reference herein. In some embodiments, an immunodeficient mouse comprises one or more alleles selected from: H2-K1^{tmlBpe}, H2-Ab1^{emlMvw}, and H2-D1^{tmlBpe}.

In some embodiments, an immunodeficient mouse model further comprises an inactivated mouse Flt3 allele. In some embodiments, an immunodeficient mouse model expresses a human FLT3L transgene. In some embodiments, an immunodeficient mouse model further comprises an inactivated mouse Flt3 allele and expresses a human FLT3L transgene, as described, for example, in WO 2020/168029, the content of which is incorporated by reference herein.

An NSG-SGM3 mouse is the NSG derivative mouse NOD.Cg-Prkdc^{scid} Il2rg^{tm1Wjl} Tg(CMV-IL3,CSF2,KITLG)1Eav/MloySzJ (Jackson Laboratory Stock No: 013062). The transgenic NSG-SGM3 mice express three human cytokines: human Interleukin-3 (IL-3), human Granulocyte/Macrophage-colony stimulating factor 2 (GM-CSF), and human Stem Cell Factor (SCF). NSG-SGM3 mice combine the features of the highly immunodeficient NSG mouse with expression of human cytokines IL-3, GM-CSF, and SCF that support stable engraftment of myeloid lineages and regulatory T cell populations.

An NSG-IL-15 mouse, NOD.Cg-Prkdc^{scid} Il2rg^{tm1Wjl} Tg(IL15)1Sz/SzJ (Jackson Laboratory Stock No: 030890), expresses human IL15 and is combined with the highly immunodeficient NOD scid gamma (NSG) mouse. Expression of human IL15, in some embodiments, enhances the development of human NK cells in immunodeficient mice engrafted with human stem cells.

Therefore, the transgenic mice described herein may be produced by breeding an immunodeficient transgenic mouse comprising a transgene encoding human interleukin-3 (IL-3), a transgene encoding human granulocyte/macrophage-colony stimulating factor 2 (GM-CSF), a transgene encoding human stem cell factor (SCF), and a transgenic mouse comprising a transgene encoding human IL-15. In some embodiments, the transgenic mice described herein are produced by breeding an NSG-SGM3 mouse with an NSG-IL-15 mouse.

### Humanized Mouse Models

Provided herein, in some embodiments, are humanized immunodeficient mouse models and methods of producing the models. Immunodeficient mice engrafted with functional human cells and/or tissues are referred to as “humanized mice.” As used herein, the terms “humanized mouse,” “humanized immune deficient mouse,” “humanized immunodeficient mouse,” and the plural versions thereof are used interchangeably to refer to an immunodeficient mouse humanized by engraftment with functional human cells and/or tissues. For example, mouse models may be engrafted with human hematopoietic stem cells (HSCs) (e.g., CD34+ HSCs) and/or human peripheral blood mononuclear cells (PMBCs). In some embodiments, mouse models are engrafted with human tissues such as islets, liver, skin, and/or solid or hematologic cancers. In other embodiments, mouse models may be genetically modified such that endogenous mouse genes are converted to human homologs (see, e.g., Pearson, et al., Curr Protoc Immunol., 2008, Chapter: Unit—15.21).

Humanized mice are generated by starting with an immunodeficient mouse (e.g., an immunodeficient mouse of 2, 3, 4, 5, 6, 7, 8, 9, 10, or more weeks of age) and, if necessary, depleting and/or suppressing any remaining murine immune cells (e.g., chemically or with radiation). That is, successful survival of the human immune system in the immunodeficient mice may require suppression of the mouse's immune system to prevent GVHD (graft-versus-host disease) rejections. After the immunodeficient mouse's immune system has been sufficiently suppressed, the mouse is engrafted with human cells (e.g., HSCs and/or PBMCs). As used herein, “engraft” refers to the process of the human cells migrating to, and incorporating into, an existing tissue of interest in vivo. With respect to the humanized immunodeficient mouse, the engrafted human cells provide functional mature human cells (e.g., immune cells). The model has a specific time window of about 4-5 weeks after engraftment before GVHD sets in. To increase the longevity of the model, double-knockout mice lacking functional MHC I and MHC II, as described above, may be used.

The engrafted human cells (e.g., HSCs or PMBCs) for humanization, in some embodiments, are human leukocyte-antigen (HLA)-matched to the human cells (e.g., human cancer cells) of the mouse models. HLA-matched refers to cells that express the same major histocompatibility complex (MHC) genes. Engrafting mice with HLA-matched human xenografts and human immune cells, for example, reduces or prevents immunogenicity of the human immune cells. In some embodiments, a humanized mouse provided in the present disclosure is engrafted with human PMBCs or human HSCs that are HLA-matched to a PDX or human cancer cell line.

The engrafted human cells (e.g., HSCs or PMBCs) for humanization, in some embodiments, are not HLA-matched to the human cells (e.g., human cancer cells) of the mouse models. That is, in some embodiments, a humanized mouse provided in the present disclosure is engrafted with human PMBCs or human HSCs that are not HLA-matched to a PDX or human cancer cell line.

### Myeloablation

As described above, in some embodiments, immunodeficient mice are treated to deplete and/or suppress any remaining murine immune cells (e.g., chemically and/or with radiation). In some embodiments, immunodeficient mice are treated only chemically or only with radiation. In other embodiments, immunodeficient mice are treated both chemically and with radiation.

In some embodiments, immunodeficient mice are administered a myeloablative agent, that is, a chemical agent that suppresses or depletes murine immune cells. Examples of myeloablative agents include busulfan, dimethyl mileran, melphalan, and thiotepa.

In some embodiments, immunodeficient mice are irradiated prior to engraftment with human cells, such as human HSCs and/or PMBCs. It is thought that irradiation of an immunodeficient mouse destroys mouse immune cells in peripheral blood, spleen, and bone marrow, which facilitates engraftment of human cells, such as human HSCs and/or PMBCs (e.g., by increasing human cell survival factors), as well as expansion of other immune cells. Irradiation also shortens the time it takes to accumulate the required number of human immune cells to “humanize” the mouse models.

For immunodeficient mice (e.g., NSG™ mice), this preparation is commonly accomplished through whole-body gamma irradiation. Irradiators may vary in size depending on their intended use. Animals are generally irradiated for short periods of time (less than 15 min). The amount of time spent inside the irradiator varies depending on the radioisotope decay charts, amount of irradiation needed, and source of ionizing energy (that is, X-rays versus gamma rays, for which a cesium or cobalt source is needed).

While a myeloablative irradiation dose is usually 700 to 1300 cGy, the data provided herein demonstrates that, in some embodiments, lower doses such as 50-200 cGy (e.g., about 50, 100, 150, or 200 cGy) may be used. As an example, the mouse may be irradiated with 50 cGy, 75 cGy, 100 cGy, 125 cGy, 150 cGy, 175 cGy, or 200 cGy.

In some embodiments, the dose is about 1, 2, 3, 4, 5, 10, 20, 100, 125, 150, 175, 200, 300, 400, 500, 600, 700, 800, 900, 1000, 1100, 1200, or 1300 cGy, or between any of the two recited doses herein, such as 50-200, 100-300 cGy, 200-500 cGy, 600-1000 cGy, or 700-1300 cGy.

In some embodiments, the immunodeficient mouse is irradiated or otherwise exposed to a myeloablative treatment about 15 minutes, 30 minutes, 45 minutes, 1 hour, or more before engraftment with human HSCs and/or PMBCs. In some embodiments, the immunodeficient mouse is engrafted with human HSCs and/or PMBCs on the same day as irradiation or other myeloablative treatment. In some embodiments, the immunodeficient mouse is engrafted with human HSCs and/or PMBCs 1, 2, 3, 4, 5, 6, 7, 8, 9, 10, 11, 12, 13, 14, 15, 16, 17, or 18 days after irradiation or other myeloablative treatment.

### Engraftment (Humanization)

As described above, in some embodiments, the irradiated immunodeficient mice are engrafted with HSCs and/or PBMCs, humanizing the mice. Engraftment refers to the process of the human cells migrating to, and incorporating into, an existing tissue of interest in vivo. The PBMCs may be engrafted after irradiation and before engraftment of human diseased cells (e.g., human cancer cells), after irradiation and concurrently with engraftment of human diseased cells, or after irradiation and after engraftment of human diseased cells.

Peripheral blood mononuclear cells (PBMCs) are peripheral blood cells having a round nucleus. These mononuclear blood cells recirculate between tissues and blood and are a critical component in the immune system to fight infection and adapt to intruders. There are two main types of mononuclear cells, lymphocytes and monocytes. The lymphocyte population of PBMCs typically includes T cells, B cells and NK cells.

PBMCs may be isolated from whole blood samples, for example (e.g., Ficoll gradient). In some embodiments, PBMCs from a subject (e.g., a human subject) with a current or previous diagnosis of cancer or an autoimmune disease may be used.

Hematopoietic stem cells (HSCs) are the stem cells that give rise to other blood cells during a process referred to as hematopoiesis. Hematopoietic stem cells give rise to different types of blood cells, in lines called myeloid and lymphoid. Myeloid and lymphoid lineages both are involved in dendritic cell formation. Myeloid cells include monocytes, macrophages, neutrophils, basophils, eosinophils, erythrocytes, and megakaryocytes to platelets. Lymphoid cells include T cells, B cells, natural killer cells, and innate lymphoid cells.

Methods of engrafting immunodeficient mice with HSCs and/or hPBMCs to yield a humanized mouse model include but are not limited to intraperitoneal or intravenous injection (Shultz et al., J Immunol, 2015, 174:6477-6489; Pearson et al., Curr Protoc Immunol. 2008; 15-21; Kim et al., AIDS Res Hum Retrovirus, 2016, 32(2): 194-2020; Yaguchi et al., Cell & Mol Immunol, 2018, 15:953-962). In some embodiments, the mouse is engrafted with 1.0×10⁶-3.0×10⁷ HSCs and/or hPBMCs. In some embodiments, the mouse is engrafted with less than 1×10⁷ HSCs and/or hPBMCs. In some embodiments, the mouse is engrafted with less than 1×10⁶ to about 5×10⁶ HSCs and/or hPBMCs. In some embodiments, the mouse is engrafted with about 2×10⁶ or 1×10⁶ HSCs and/or hPBMCs. In some embodiments, the mouse is engrafted with 25,000-100,000 HSCs and/or hPBMCs (e.g., 25,000, 30,000, 35,000, 40,000, 45,000, 50,000, 55,000, 60,000, 65,000, 70,000, 75,000, 80,000, 85,000, 90,000, 95,000, 100,000 or more HSCs and/or hPBMCs).

For example, the mouse may be engrafted with 1×10⁵-1×10⁶, 1×10⁵-1×10⁷, 1×10⁵-1×10⁸, 1×10⁶-1×10⁷, 1×10⁶-1×10⁸ hPBMCs. In some embodiments, the mouse is engrafted with 1×10⁶-2×10⁶, 1×10⁶-3×10⁶, 1×10⁶-4×10⁶, 1×10⁶-5×10⁶, 1×10⁶-6×10⁶, 1×10⁶-7×10⁶, 1×10⁶-8×10⁶, 1×10⁶-9×10⁶, 2×10⁶-3×10⁶, 2×10⁶-4×10⁶, 2×10⁶-5×10⁶, 2×10⁶-6×10⁶, 2×10⁶-7×10⁶, 2×10⁶-8×10⁶, 2×10⁶-9×10⁶, 3×10⁶-4×10⁶, 3×10⁶-5×10⁶, 3×10⁶-6×10⁶, 3×10⁶-7×10⁶, 3×10⁶-8×10⁶, or 3×10⁶-9×10⁶ hPBMCs.

In some embodiments, the mouse is engrafted with 1×10⁵-1×10⁶, 1×10⁵-1×10⁷, 1×10⁵-1×10⁸, 1×10⁶-1×10⁷, 1×10⁶-1×10⁸ HSCs. In some embodiments, the mouse is engrafted with 1×10⁶-2×10⁶, 1×10⁶-3×10⁶, 1×10⁶-4×10⁶, 1×10⁶-5×10⁶, 1×10⁶-6×10⁶, 1×10⁶-7×10⁶, 1×10⁶-8×10⁶, 1×10⁶-9×10⁶, 2×10⁶-3×10⁶, 2×10⁶-4×10⁶, 2×10⁶-5×10⁶, 2×10⁶-6×10⁶, 2×10⁶-7×10⁶, 2×10⁶-8×10⁶, 2×10⁶-9×10⁶, 3×10⁶-4×10⁶, 3×10⁶-5×10⁶, 3×10⁶-6×10⁶, 3×10⁶-7×10⁶, 3×10⁶-8×10⁶, or 3×10⁶-9×10⁶ HSCs.

In some embodiments, the mouse is engrafted with a dose of less than 1×10⁷ hPBMCs (e.g., 1×10⁶ to about 5×10⁶ hPBMCs, 1×10⁶ to about 4×10⁶ hPBMCs, 1×10⁶ to about 3×10⁶ hPBMCs, about 5×10⁶ hPBMCs, about 4×10⁶ hPBMCs, about 3×10⁶ hPBMCs, about 2×10⁶ hPBMCs, or about 1×10⁶ hPBMCs).

In some embodiments, the mouse is engrafted with a dose of less than 1×10⁷ HSCs (e.g., 1×10⁶ to about 5×10⁶ HSCs, 1×10⁶ to about 4×10⁶ HSCs, 1×10⁶ to about 3×10⁶ HSCs, about 5×10⁶ HSCs, about 4×10⁶ HSCs, about 3×10⁶ HSCs, about 2×10⁶ HSCs, or about 1×10⁶ HSCs).

As described herein, in some embodiments, engraftment with HSCs and/or PBMCs yields a transgenic mouse comprising more human CD45+ cells in peripheral blood, relative to a humanized NSG-SGM3 control mouse (e.g., an increase of 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40% or more). In embodiments, the transgenic mouse comprises significantly more human NK cells in peripheral blood, relative to a humanized NSG-SGM3 control mouse (e.g., an increase of 0.5%, 1%, 2%, 3%, 4%, 5%, 6%, 7%, 8%, 9%, 10%, or more, measured as a percentage of CD45+ cells).

In some embodiments, engraftment with PBMCs yields a transgenic mouse comprising more human myeloid cells in peripheral blood relative to a humanized NSG-SGM3 control mouse (e.g., an increase of 0.01%, 0.02%, 0.03%, 0.04%, 0.05%, 0.06%, 0.07%, 0.08%, 0.09%, 0.1%, 0.11%, 0.12%, 0.13%, 0.14%, 0.15%, 0.16%, 0.17%, 0.18%, 0.19%, 0.2% or more, measured as a percentage of CD45+ cells).

In some embodiments, the transgenic mouse comprises more activated human T cells relative to a humanized NSG-SGM3 control mouse. In some embodiments, the activated T cells are present in the spleen of the transgenic mouse. The term “T-cell activation” refers to the mechanisms of activation of T-cells which may vary slightly between different types of T cells. The “two-signal model” in CD4+ T cells, however, is applicable for most types of T-cells. In more detail, activation of CD4+ T cells typically occurs through the engagement of both the T cell receptor and CD28 on the T cell surface by the major histocompatibility encoded antigen-presenting molecule and with its bound antigenic peptide and B7 family members on the surface of an antigen presenting cell (APC), respectively. Both cell-cell contacts are generally required for the production of an effective immune response. For example, in the absence of CD28 co-stimulation, T-cell receptor signaling alone may result in T-cell anergy. The further signaling pathways downstream from both CD28 and the T cell receptor involve many further proteins known to the skilled person. The activation of T-cells may be determined by cytokine release and/or cell proliferation, in particular, proliferation of T-cells.

### Human Immune Cells

The mouse models provided herein support expansion, development, and maturation of a complete repertoire of human immune cells, including cells of the innate immune system and cells of the adaptive immune system. There are two main lineages of human immune cells: the lymphoid lineage and the myeloid lineage. Progenitor cells of the lymphoid lineage develop into B cell progenitors, natural killer cells, and T cell progenitors. B cell progenitors continue to develop into either memory B cells or plasma cells, while T cell progenitors continue to develop into memory T cells, cytotoxic T cells, or helper T cells. Progenitor cells of myeloid lineage develop into leukocytes (e.g., neutrophils, eosinophils, basophils, lymphocytes, and monocytes). The monocytes further develop into dendritic cells (e.g., cDC1s or cDC2s) and macrophages.

The human immune system includes the innate immune system and the adaptive immune system. The innate immune system includes the mononuclear phagocyte system of macrophages, dendritic cells, and monocytes, natural killer cells, mast cells, γδ T cells, natural killer T cells, and granulocytes (basophils, eosinophils, and neutrophils). The adaptive immune system includes humoral immunity (also referred to as B cell immunity), which includes B cells that produce antibodies, and cellular immunity (also referred to as T cell immunity), which includes CD4+ and CD8+ T cells, natural killer cells, and γδ T cells.

T cells are key actors of the adaptive immune system, are commonly identified by CD3 expression, and detect antigen through T cell receptors (TCRs), which recognize peptides presented by the major histocompatibility complex (MHC). Circulating tumor cell antigens are delivered to lymph nodes, where they are displayed to CD4+ and CD8+ T cells, also known as T helper and cytotoxic T cells, respectively. Following activation, T helper cells release a variety of cytokines, including IFNγ. Cytotoxic T cells recognize cells expressing tumor-specific antigens and kill them through perforin- or granzyme-induced apoptosis.

Macrophages are also cells of the innate immune system and are identified by expression of CD68 and MHCII and lack of CD11c. They specialize in phagocytosis and secrete cytokines that influence the immune response. Macrophages generally are classified as pro-inflammatory (M1-like) or anti-inflammatory (M2-like). M1-like macrophages are identified by expression of CD80, CD86, or iNOS and promote the antitumor immune response by phagocytosis of malignant cells and production of T cell-activating ligands. Conversely, M2-like macrophages are identified by expression of CD163 or CD206 and can promote tumor growth through secretion of immunosuppressive cytokines, such as IL-10, and by promoting a Th2 response. M2 macrophages can also express the immunosuppressive enzyme arginase, which depletes arginine from the tumor microenvironment, leading to reduced T cell proliferation and function.

Natural killer (NK) cells represent the primary innate immune cell type. They recognize and kill cancer by detecting downregulation of MHC class I on tumor cells and/or by detecting upregulation of ligands on tumor cells that bind to activating receptors on NK cells. NK cells are commonly identified by a combination of CD56 and CD16 and lack of CD3 expression.

Some aspects of the present disclosure provide mouse models that support expansion, development, and/or maturation of cells of the adaptive immune system and cells of the innate immune system. In some embodiments, a mouse model supports expansion, development, and/or maturation of human CD33+ myeloid cells. In some embodiments, a mouse model supports expansion, development, and/or maturation of human CD45+ immune cells. In some embodiments, a mouse model supports expansion, development, and/or maturation of human CD19+ B cells (e.g., human B cells that produce circulating immunoglobulin (Ig)). In some embodiments, a mouse model supports expansion, development, and/or maturation of human CD3+ T cells. In some embodiments, a mouse model supports expansion, development, and/or maturation of human NK cells.

### B Cell Antibody Class Switching

The mouse models provided herein, in some embodiments, support expansion of mature human B cells that are capable of immunoglobulin (Ig) class switching, which is a biological mechanism that changes a B cell's production of Ig from one type to another, such as from the isotype IgM to the isotype IgG. Class switching occurs after activation of a mature B cell via its membrane-bound antibody molecule (or B cell receptor) to generate the different classes of antibody, all with the same variable domains as the original antibody generated in the immature B cell during the process of V(D)J recombination, but possessing distinct constant domains in their heavy chains.

The term “Ig,” as used herein, refers to a region of an immunoglobulin that exists as a distinct structural entity as ascertained by one skilled in the art of protein structure. Ig domains typically have a characteristic β-sandwich folding topology. There are five different classes of antibodies in humans, including IgA (which comprises subclasses IgA1 and IgA2), IgD, IgE, IgG (which comprises subclasses IgG1, IgG2, IgG3, and IgG4), and IgM. Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. The distinguishing feature between these antibody classes is their constant regions, although subtler differences may exist in the V region.

The term “IgG,” as used herein, refers to a polypeptide belonging to the class of antibodies that are substantially encoded by a recognized immunoglobulin gamma gene. In humans, this class comprises the subclasses or isotypes IgG1, IgG2, IgG3, and IgG4. IgG antibodies are tetrameric proteins composed of two heavy chains and two light chains. The IgG heavy chain comprises four immunoglobulin domains linked from N- to C-terminus in the order VH-CH1-CH2-CH3, referring to the heavy chain variable domain, heavy chain constant domain 1, heavy chain constant domain 2, and heavy chain constant domain 3 respectively (also referred to as VH-C.gamma.1-C.gamma.2-C.gamma.3, referring to the heavy chain variable domain, constant gamma 1 domain, constant gamma 2 domain, and constant gamma 3 domain respectively). The IgG light chain is composed of two immunoglobulin domains linked from N- to C-terminus in the order VL-CL, referring to the light chain variable domain and the light chain constant domain, respectively.

Naïve mature B cells produce both IgM and IgD, which are the first two heavy chain segments in the immunoglobulin locus. After activation by antigen, these B cells proliferate. If these activated B cells encounter specific signaling molecules via their CD40 and cytokine receptors (both modulated by T helper cells), they undergo antibody class switching to produce IgG, IgA or IgE antibodies. During class switching, the constant region of the immunoglobulin heavy chain changes but the variable regions do not, and therefore antigenic specificity remains the same. This allows different daughter cells from the same activated B cell to produce antibodies of different subclasses or isotypes.

In some embodiments, the amount of IgG produced in the mouse models provided herein is within the physiological range found in humans, wherein the normal physiological range for IgG in adult humans is 6000-16000 μg/mL.

In some embodiments, the mouse models provided herein produce subclasses of IgG (i.e., IgG1, IgG2, IgG3, and/or IgG4). In some embodiments, the amount of IgG1, IgG2, IgG3, and/or IgG4 produced are within the physiological range found in humans, wherein the normal physiological ranges for IgG subclasses in adult humans are as follows: 2.0-8.00 mg/mL IgG1; 1.15-5.70 mg/mL IgG2; 0.24-1.25 mg/mL IgG3; and 0.052-1.25 mg/mL IgG4.

In some embodiments, the amount of IgM and/or IgA produced in the mouse models provided herein are within the physiological range found in humans, wherein the normal physiological range of in adult humans is 0.4-2.5 mg/mL IgM and 0.8-3.0 mg/mL IgA.

### Mammalian Cell Lines and Patient-Derived Xenografts

The mouse models of disease provided herein, in some embodiments, are engrafted with tissue or cells (viable cells), for example, mammalian cells, such as those from a cell line or a patient (e.g., a human or canine patient-derived xenograft). As used herein, “mammal” includes, but is not limited to, mice, rats, rabbits, guinea pigs, dogs, cats, sheep, goats, cows, horses, primates such as monkeys, chimpanzees and monkeys, and, in particular, humans. In some embodiments, a mouse model is engrafted with human tissue (cells). In other embodiments, a mouse model is engrafted with canine cells.

The cells may be diseased cells, in some embodiments, for example, cancer cells, cells involved in autoimmunity, or cells involved in other inflammatory diseases. Other diseased cells are contemplated herein (e.g., those cells obtained from a patient having a cardiovascular disease, metabolic disease, etc.).

In some embodiments, the mouse models are engrafted with human cancer cells. The human cancer cells may be from a single source or from multiple sources (e.g., at least 2, at least 3, at least 4, at least 5, at least 6, at least 7, at least 8, at least 9, or at least 10 sources). The human cancer cells may be tumor cells (e.g., cells from a malignant tumor), patient-derived xenografts (PDXs) (e.g., tumor tissue from a human that is implanted in a mouse model), or human cancer cell lines (e.g., human cancer cell cultures developed from a single cell). A tumor is a mass of tissue formed by the abnormal growth of cells (e.g., cancer cells). Non-limiting examples of common human cancers include bladder cancer, brain cancer, breast cancer, colon and rectal cancer, endometrial cancer, kidney cancer, leukemia, liver cancer, lung cancer, melanoma, non-Hodgkin lymphoma, ovarian cancer, pancreatic cancer, prostate cancer, sarcoma, skin cancer, testicular cancer, and thyroid cancer. Other cancer cell types are contemplated herein (see, e.g., cancer.gov/types).

In some embodiments, human cancer cells may be circulating tumor cells, for example, from a primary tumor or a secondary tumor. A primary tumor is a tumor growing at the anatomical site where the tumor originated (e.g., lung cancer tumor or breast cancer tumor). A secondary tumor is a tumor that is the same type as a primary tumor (e.g., lung cancer or breast cancer) but has formed at a secondary anatomical site that is separate from the primary tumor.

In some embodiments, the mouse models are engrafted with a patient-derived xenograft (PDX). A PDX is a tumor tissue from a human or other mammal that is implanted in a mouse model of the present disclosure, for example. A PDX used herein may be obtained directly from a subject or obtained from a PDX repository. Non-limiting examples of PDX repositories include Jackson Laboratories Mouse Models of Human Cancer Database (Krupke, D M, et al., “Mouse Models of Human Cancer Database,” Nat Rev Cancer, 2008 8(6): 459-65), Dana Farber Cancer Institute Patient-Derived Tumor Xenograft Database, and Charles River Patient-Derived Xenograft Model Database. A model of disease may comprise at least 2 (e.g., at least 3, at least 4, at least 5, at least 6, at least 7, at least 8, at least 9, or at least 10) PDXs.

A PDX may have any human tumor origin. For example, a PDX may be from bladder tumor, brain tumor, breast tumor, colon and rectal tumor, endometrial tumor, kidney tumor, leukemia, liver tumor, lung tumor, melanoma, non-Hodgkin lymphoma, ovarian cancer, pancreatic tumor, prostate tumor, sarcoma, skin cancer, testicular cancer, or thyroid tumor. Other tumor types are contemplated herein (see, e.g., cancer.gov/types).

In some embodiments, the mouse models are engrafted with human cancer cells from a human cancer cell line. Human cancer cell lines are human cancer cell cultures developed from a single cell. In some embodiments, a human cancer cell line is immortalized. Immortalized cells divide and proliferate indefinitely. Human cancer cell lines may be from any human cancer. For example, a human cancer cell line may be from a bladder tumor (HTB-9, HTB-3, CRL-2169), breast tumor (e.g., Hs.281.T, Hs 5788st, UACC-812, MCF 10A, or MDA-MB-157), brain tumor (SW 1088, U138, Daoy, LN-228), colon and rectal tumor (HT29, SW480, SW 1116, Caco-2), endometrial tumor (Ishikawa), kidney tumor (Caki-1, 769-P), leukemia (MOLT-3, TALL-104, AML-193, Jurkat, Mo-B), liver tumor (e.g., SNU-182, Hs.817.T, NCI-H735, or THLE-3), lung tumor (e.g., NCI-H838, HCC827, NCI-H1666, SW 1573, ChaGo-K-1, A549, or NCI-H1555), melanoma (SK-MEL-3, A375-P, MNT-1), non-Hodgkin lymphoma (e.g., GA-10, NCI-BL2171, HH, or Toledo), ovarian tumor (SK-OV-3, PA-1, Caov-3, SW 636), pancreatic tumor (Capan-2, Panc 10.05, CFPAC-1, SQ 1990), prostate tumor (VCaP, C4-2B, LNCaP, PC-3), sarcoma (HS 822.T, SK-LMS-1, A-204), skin (TE 354.T, Hs 456.Bt,), testicular (Cates-1B, Hs 1.Tes), or thyroid tumor (MDA-T120, MDA-T41, SW-579). A mouse model herein may be engrafted with human cancer cell lines from a single cell line or from multiple cell lines (e.g., at least 2, at least 3, at least 4, at least 5, at least 6, at least 7, at least 8, at least 9, or at least 10 cell lines).

Further, any of the mouse models provided herein may include a combination of human cancer cell types, optionally from a combination of sources, e.g., PDX sources and/or cell lines.

In some embodiments, human cancer cells are genetically engineered to express a detectable biomolecule, for example, so that the cells can be monitored in vivo or analyzed ex vivo. A detectable biomolecule, as provided herein, refers to a biomolecule in a human cancer cell that can be detected using a conventional or non-conventional assay. Non-limiting examples of detectable biomolecules include fluorescent proteins (e.g., green fluorescent protein, yellow fluorescent protein, or red fluorescent protein), antigens (e.g., hemagglutinin or human leukocyte antigen), and enzymes (e.g., beta-galactosidase or luciferase). Other detectable biomolecules are contemplated herein.

Methods for obtaining human cancer cells and PDXs include but are not limited to biopsy (e.g., hollow-needle, excisional, incisional, or brush), excision (e.g., of a tumor), and collection of a sample (e.g., blood sample) followed by a sorting step to isolate human cancer cells from other cells (e.g., human non-cancer cells, non-human cells, or cell fragments).

In some embodiments, the mouse models are engrafted with human cells from a subject having an autoimmune disease. Autoimmune diseases arise from and are directed against, a subject's own tissues. Examples of autoimmune diseases include, but are not limited to arthritis (rheumatoid arthritis, juvenile rheumatoid arthritis, osteoarthritis, psoriatic arthritis, and ankylosing spondylitis), psoriasis, dermatitis including atopic dermatitis; chronic idiopathic urticaria, including chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), and IBD with co-segregate of pyoderma gangrenosum, erythema nodosum, primary sclerosing cholangitis, and/or episcleritis), respiratory distress syndrome, including adult respiratory distress syndrome (ARDS), meningitis, IgE-mediated diseases such as anaphylaxis and allergic rhinitis, encephalitis such as Rasmussen's encephalitis, uveitis, colitis such as microscopic colitis and collagenous colitis, glomerulonephritis (GN) such as membranous GN, idiopathic membranous GN, membranous proliferative GN (MPGN), including Type I and Type II, and rapidly progressive GN, allergic conditions, eczema, asthma, conditions involving infiltration of T cells and chronic inflammatory responses, atherosclerosis, autoimmune myocarditis, leukocyte adhesion deficiency, systemic lupus erythematosus (SLE) such as cutaneous SLE, lupus (including nephritis, cerebritis, pediatric, non-renal, discoid, alopecia), juvenile onset diabetes, multiple sclerosis (MS) such as spino-optical MS, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including Large Vessel vasculitis (including Polymyalgia Rheumatica and Giant Cell (Takayasu's) Arteritis), Medium Vessel vasculitis (including Kawasaki's Disease and Polyarteritis Nodosa), CNS vasculitis, and ANCA-associated vasculitis, such as Churg-Strauss vasculitis or syndrome (CSS), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory diseases, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, and rejection arising from renal transplantation, liver transplantation, intestinal transplantation, cardiac transplantation, etc.), graft versus host disease (GVHD), pemphigoid bullous, pemphigus (including vulgaris, foliaceus, and pemphigus mucus-membrane pemphigoid), autoimmune polyendocrinopathies, Reiter's disease, stiff-man syndrome, immune complex nephritis, IgM polyneuropathies or IgM mediated neuropathy, idiopathic thrombocytopenic purpura (ITP), thrombotic throbocytopenic purpura (TTP), thrombocytopenia (as developed by myocardial infarction patients, for example), including autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM), including pediatric IDDM, and Sheehan's syndrome; autoimmune hepatitis, Lymphoid interstitial pneumonitis (HIV), bronchiolitis obliterans (non-transplant) vs NSIP, Guillain-Barré Syndrome, Berger's Disease (IgA nephropathy), primary biliary cirrhosis, celiac sprue (gluten enteropathy), refractory sprue with co-segregate dermatitis herpetiformis, cryoglobulinemia, amylotrophic lateral sclerosis (ALS; Lou Gehrig's disease), coronary artery disease, autoimmune inner ear disease (AIED), autoimmune hearing loss, opsoclonus myoclonus syndrome (OMS), polychondritis such as refractory polychondritis, pulmonary alveolar proteinosis, amyloidosis, giant cell hepatitis, scleritis, monoclonal gammopathy of uncertain/unknown significance (MGUS), peripheral neuropathy, paraneoplastic syndrome, channelopathies such as epilepsy, migraine, arrhythmia, muscular disorders, deafness, blindness, periodic paralysis, and channelopathies of the CNS; autism, inflammatory myopathy, and focal segmental glomerulosclerosis (FSGS).

The humanized immunodeficient mouse model of disease provide herein are engrafted with human cells, for example, human PBMCs, human HSCs, and/or human cancer cells from a human cancer cell line or human PDX, as discussed above. Engraftment refers to the process of the human cells migrating to, and incorporating into, an existing tissue of interest in vivo.

Any tissue in a mouse may be the target tissue for engraftment of the human cells (e.g., human PBMC, HSC, or cancer cells). The target tissue for engraftment is the tissue to which the human cells (e.g., human PBMC, HSC, or cancer cells) will migrate and incorporate. The target tissue for engraftment may depend on the disease to be studied. Non-limiting examples of target tissues for engraftment of human cells (e.g., human PBMC, HSC, or cancer cells) include lung, trachea, liver, bone marrow, brain, blood, gastrointestinal tissue, skin, stomach, small intestine, large intestine, and pancreas. Other target tissues are contemplated herein. In some embodiments, human cells will engraft in one target tissue, while in some embodiments, human cells will engraft in more than one (e.g., 2, 3, 4, 5, 6, 7, 8, 9, 10 or more) target tissue.

In some embodiments, the human cells (e.g., human PBMC, HSC, or cancer cells) are delivered to a mouse using a single cell suspension. A single-cell suspension is a suspension of cells that lacks detectable levels of cell debris and cell aggregates. Single-cell suspensions enable separation of cells from tissues (e.g., connective tissue) and maximize the efficiency of using human cells, including, but not limited to: engraftment into a model animal (e.g., mouse model), flow cytometry, human cell culture (e.g., immortalization), and human cell labeling. Methods for preparing human cell single-cell suspensions depend on the origin of the cells (e.g., freshly isolated from human, derived from PDX, cell lines). Methods for preparing single-cell suspensions include but are not limited to dissociation (e.g., enzymatic, mechanical), purification (e.g., magnetic activated cell sorting or activated cell sorting), commercial kits (e.g., Miltenyi Biotec or StemCell), centrifugation (e.g., at >300×g), and filtering (e.g., cell strainer).

For example, the mouse may be engrafted with 1.0×10⁵-2.0×10⁷ human cells (e.g., human PBMC, HSC, or cancer cells). In some embodiments, the mouse is engrafted with 1.0×10⁵, 2.0×10⁵, 3.0×10⁵, 4.0×10⁵, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, or 2.0×10⁷ human cells (e.g., human PBMC, HSC, or cancer cells).

The human cells (e.g., human PBMC, HSC, or cancer cells) may be delivered to a mouse via injection (e.g., tail vein, retroorbital, intravenous, intracardiac, or intraarterial) or implantation (e.g., subcutaneous, intraperitoneal, intrafemoral, intratibial, or intramuscular). In some embodiments, the delivery is via subcutaneous implantation. Other delivery methods are contemplated herein.

As described herein, in some embodiments, engraftment with HSCs or PBMCs and a xenograft (e.g., human xenograft tissue or cells) yields a transgenic mouse comprising fewer human regulatory T (Treg) cells in peripheral blood, relative to a humanized NSG-SGM3 control mouse (e.g., a decrease of 1%, 2%, 3%, 4%, 5%, 6%, 7%, 8%, 9%, 10%, 11%, 12%, 13%, 14%, 15%, 16%, 17%, 18%, 19%, 20%, 25%, 30%, or more, measured as a percentage of CD45+ cells). In some embodiments, the transgenic mouse comprises fewer PD-1+ T cells (CD4 and/or CD8) cells in peripheral blood, relative to a humanized NSG-SGM3 control mouse (e.g., a decrease of 1%, 2%, 3%, 4%, 5%, 6%, 7%, 8%, 9%, 10%, 11%, 12%, 13%, 14%, 15%, 16%, 17%, 18%, 19%, 20%, 25%, 30% 35%, 40%, 45%, 50%, 55%, 60%, 65%, 70%, or more, measured as a percentage of CD4 and/or CD8 T cells).

In some embodiments, the exponential growth phase of the tumor is delayed; that is, it begins 30, 35, 36, 37, 38, 39, 40, 41, 42, 43, 44, 45, 50, or more days after implantation of the tumor.

### Methods of Use

A humanized, transgenic, immunodeficient mouse model of disease provided herein may be used for any number of applications. For example, a humanized, immunodeficient model (e.g., immunodeficient mouse model) may be used to assess disease development, to assess disease progression, and/or test how a candidate prophylactic agent or a therapeutic agents (e.g., candidate therapeutic agent) affects the human immune system (e.g., whether it elicits cytokine release syndrome).

### Assessment of Disease Behavior

Immunodeficient models (e.g., mouse models) provided herein may be used to assess disease behavior. Disease behavior refers to the changes that occur in a host (e.g., immunodeficient mouse model) due to disease (e.g., cancer, autoimmunity, and/or inflammation). Non-limiting examples of disease behavior include spread of disease to multiple tissues (e.g., metastasis), production of symptoms of disease, and production of critical genes and/or proteins for disease progression.

Provided herein, in some embodiments, are methods of assessing spread of disease to multiple tissues in a host (e.g., immunodeficient mouse model). Multiple tissues may be multiple examples of the same type of tissue (e.g., lung tissue) or multiple different tissue types (e.g., lung tissue, blood, liver, or brain). Any method may be used to assess spread of disease including, but not limited to: obtaining multiple tissue samples and verifying (e.g., immunohistochemistry) presence of diseased cells and/or tissue; and performing in vivo imaging system (IVIS) (e.g., with a fluorescent marker).

Provided herein, in some embodiments, are methods of assessing production of symptoms of disease and disease progression. The symptoms of disease and disease progression may depend on the disease and the host (e.g., humanized immunodeficient mouse model). It is desirable that symptoms of disease and disease progression in models provided in the present disclosure mimic symptoms of disease and disease progression in humans. Non-limiting examples of symptoms of disease and disease progression include fever, lethargy, weight loss, diarrhea, muscle aches, coughing, difficulty breathing, vomiting, seizures, ataxia, change in blood pressure, proteinuria, hematuria, loss of fur, edema, erythema, dermatitis, and dehydration. Any method may be used to assess production of symptoms of disease and disease progression. Non-limiting examples of methods used to assess production of symptoms of disease and disease progression include measuring temperature, monitoring sleep/wake cycles, monitoring activity, measuring weight, assessing solid and liquid excrement, monitoring breathing and assessing lung function, monitoring blood pressure and cardiac function, measuring protein or blood presences in the urine, measuring cytokine levels in the blood, evaluating changes in skin or mucous membrane thickness, measuring ion concentration in blood, and any disease activity index (DAI) evaluation.

### Assessment of Disease Impact on Human Immune System

Humanized immunodeficient mouse models provided herein may be used to assess disease impact on the human immune system. Non-limiting examples of disease impact on the human immune system include modulated human immune cell production and cytokine release.

Provided herein, in some embodiments, are methods of assessing modulated human immune cell production. Modulated human immune cell production may be increased human immune cell production (e.g., compared to a control) or decreased human immune cell production (e.g., compared to a control). A control may be a humanized immunodeficient model (e.g., mouse model) without the disease. The production of any human immune cell in a humanized immunodeficient model (e.g., mouse models) may be modulated by disease. Non-limiting examples of human immune cells whose production may be modulated by disease include: hematopoietic stem cells (e.g., surface marker CD34+), T-cells (e.g., surface markers CD3+, CD4+, CD8+), B cells (e.g., surface marker CD19+, CD20+), natural killer cells, plasma cells, immunoglobulins, neutrophils, monocytes, dendritic cells, and cytokines (e.g., IL-2, IL-4, or IL-6).

In some embodiments, the mouse model may be used to assess cytokine release syndrome (CRS), a systemic inflammatory response in a subject inter alia characterized by hypotension, pyrexia and/or rigors, and potentially resulting in death. A cytokine storm (CRS) is presumably caused by an uncontrolled positive feedback loop between cytokines and immune cells, resulting in highly elevated levels of various cytokines. While these terms may differ some in degree, they are all the result of unacceptably high release of cytokines by a subject, as a result of administration of certain immunomodulatory drugs, e.g., antibodies, to the subject. The subject reacts to the treatment by releasing the unacceptably high levels of cytokine. The humanized mouse model described herein may be used as a drug testing platform to identify from a large number of clinically relevant drug candidates the potential drug candidates that elicit cytokine release. The present methods therefore represent robust prediction assays for drug immunotoxicity testing, providing a link between pre-clinical and clinical testing. Therefore, in one aspect, the present disclosure is directed to a method of determining whether an immunomodulatory drug causes immune toxicity in a human. In some embodiments, levels of specific cytokines (e.g., IFN-γ, IL-2, IL-4, IL-6, IL-10, and/or TNF) are measured as part of the assessment of a drug candidate's potential immunotoxicity.

Any method of measuring human immune cell production may be used to assess modulated human immune cell production. Non-limiting examples of methods of measuring human immune cell production include flow cytometry, fluorescence activated cell sorting (FACS), RT-PCR of human immune cell surface markers and cytokines, and ELISA.

Some aspects provide method of using the mouse models described herein to assess anti-drug antibodies (ADA) with human therapeutics. ADA may induce unwanted side effects, especially in biotechnology-derived pharmaceuticals, such as therapeutic antibodies and growth factors. Thus, ADA have been subjected to increasing scrutiny by the regulatory authorities using immunogenicity safety studies. ADA have been observed in preclinical and clinical studies resulting in significant changes in toxicology, pharmacokinetics, and efficacy. These effects result from the generation of drug-induced (neutralizing) autoantibodies against, for example, erythropoietin (EPO) Factor VIII (FVIII), or insulin and can be responsible for allergic reactions, or even anaphylactic shock. As a consequence, studies on ADA have become inevitable for bioengineered pharmaceuticals including biosimilars. Adverse immunological reactions may vary widely, depending on how the active ingredients are structured, produced and applied. For example, the expression of anti-Fc antibodies, anti-idiotypic antibodies or antibodies against glycosylated antigens may appear. The detection and characterization assays for ADA must therefore be developed, customized and optimized for each drug.

The humanized immunodeficient mouse models provided herein, in some aspects, are used as in vivo models to assess ADA, for example, long term development of ADA.

In some embodiments, a mouse model used to assess ADA undergoes a myeloablative procedure, such as irradiation or chemical ablation.

In some embodiments, a mouse model used to assess ADA is engrafted with huPBMCs (e.g., target drug naïve huPBMC), as discussed elsewhere herein.

Following engraftment, a target drug may then be administered. The term “target drug” encompasses human therapeutic modalities (e.g., agents) that elicit a humoral response. Non-limiting examples include therapeutic antibodies, recombinant protein therapeutics (e.g., growth factors), cell-based therapies (e.g., chimeric antigen receptor (CAR)-T cell, TCR-engineered T cell, tumor-infiltrating lymphocyte (TIL), and regulatory T cell (Treg) therapies), DNA-based (e.g., gene, antisense oligonucleotide) therapies, and RNA-based (e.g., RNAi and mRNA) therapies.

In some embodiments, a target drug is administered to the mouse about 4 to 10 days following administration of the hPBMCs. For example, a target drug may be administered 4, 5, 6, 7, 8, 9, or 10 days following administration of the hPBMCs. In some embodiments, a target drug is administered to the mouse at least 4 days, at least 5 days, at least 6 days, at least 7 days, at least 8 days, at least 9 days, or at least 10 days following administration of the hPBMCs. In some embodiments, a single dose of the target drug is administered. In other embodiments, multiple doses (e.g., 2-10, e.g., 2, 3, 4, 5, 6, 7, 8, 9, or 10) doses of the target drug are administered (e.g., weekly, every other week, every three weeks, or monthly). In some embodiments, a target drug is administered according to a standardized dosing schedule.

Anti-drug antibodies may be assessed over a period of time, for example, about 7 to about 270 days. In some embodiments, ADA are assessed by collecting a blood sample from the mouse and characterizing the plasma and/or B cell function. In some embodiments, flow cytometry or ELISA assays are used to assess the blood sample (or other biological sample) for antibody recognition and/or neutralization of the target drug. An initial bleed, prior to target drug administration, may serve as a control, for example. In some embodiments, anti-drug Ig levels increase over time.

An analysis of ADA may include a characterization of ADA titer, neutralizing capacity, binding affinity, isotyping, and other characteristics. There are several isotypes of ADA. For example, IgM ADA may be an early marker of ADA formation, and the presence of IgE antibodies may indicate an allergic reaction against the target drug. In addition, the measurement of IgG subclasses may be supportive for the biological activities of ADA since in humans, IgG1 and IgG3 are mainly involved in complement activation and are more prone to NK cell recognition. The measurement of binding affinities of ADA is also informative for the ADA response interpretation.

### Assessment of Prophylactic and Therapeutic Agents

Immunodeficient models (e.g., mouse models) provided in the present disclosure may be used to assess prophylactic agents and therapeutic agents for preventing or treating disease or disease progression. A prophylactic agent is a substance (e.g., drug or protein) that prevents or reduces risk of disease or prevents or reduces the risk of the development of disease (disease progression). A therapeutic agent is a substance (e.g., drug or protein) that treats a disease (e.g., treats symptoms associated with the disease). Therapeutic agents include palliative agents, which are substances (e.g., drug or protein) that ameliorates one or more symptoms of a disease.

In some embodiments, the humanized transgenic mouse is administered an agent that activates human T cells. In some embodiments, the transgenic mouse is then administered a therapeutic agent, such as an anti-cancer agent or an anti-autoimmune disease agent (e.g., an anti-inflammatory agent, a corticosteroid, or an immunosuppressive agent). In further embodiments, the efficacy of the therapeutic agent is measured.

With respect to prevention of a disease, it should be understood that a prophylactically effective amount of an agent need not entirely eradicate the disease but should reduce or prevent the progression of the disease (e.g., metastasis). In some embodiments, a prophylactically effective amount of an agent reduces disease progression in the subject by at least 30%, at least 40%, at least 50%, at least 60%, at least 70%, at least 80%, or at least 90%. It should be understood that a therapeutic agent (e.g., a palliative agent) need not impact the diseased tissues or cells but should alleviate at least one symptom of the disease and thus potentially mitigate the short- or long-term systemic impact of the disease.

A prophylactic agent and/or a therapeutic agent may be delivered by any method. Non-limiting examples of methods of delivering a prophylactic agent and/or a therapeutic agent include: inhalation (e.g., nasal or tracheal), injection (e.g., intravenous, intraarterial, intramuscular, or intracranial), and ingestion (e.g., tablet or liquid).

In some embodiments, the agent is a vaccine, such as a human vaccine. The vaccine may be a cancer vaccine or an infectious disease vaccine, for example. In some embodiments, the agent is a protein antigen, for example, from a pathogen, such as a virus or bacteria. In other embodiments, the agent is a nucleic acid. Non-limiting examples of nucleic acid vaccines include RNA (e.g., mRNA) or DNA encoding a protein antigen.

In some embodiments, a candidate agent is an analgesic, anti-pyretic, anti-inflammatory drug, or an immunosuppressive including but not limited to NSAIDs, steroids, diuretics, statins, and beta-blockers.

Combinations of any of the prophylactic agents and/or therapeutic agents provided herein may also be administered to an immunodeficient model (e.g., mouse) inoculated with diseased cells and/or tissue. In some embodiments, an immunodeficient model with diseased cells and/or tissue is administered 1 or more, 2 or more, 3 or more, 4 or more, 5 or more, 6 or more, 7 or more, 8 or more, 9 or more, or 10 or more prophylactic agents. In some embodiments, an immunodeficient model (e.g., mouse) with diseased cells and/or tissue is administered 1 or more, 2 or more, 3 or more, 4 or more, 5 or more, 6 or more, 7 or more, 8 or more, 9 or more, or 10 or more therapeutic (e.g., palliative) agents. In some embodiments, an immunodeficient model (e.g., mouse) with diseased cells and/or tissue is administered 1 or more, 2 or more, 3 or more, 4 or more, 5 or more, 6 or more, 7 or more, 8 or more, 9 or more, or 10 or more prophylactic and therapeutic agents.

Any effective amount of a prophylactic agent and/or a therapeutic agent may be administered to a subject (e.g., immunodeficient model or human patient). An effective amount is a dose (e.g., mg, mg/mL, or mg/kg) that prevents or reduces the risk of disease or disease progression and/or treats the disease. Any dosage regimen may be used to administer a prophylactic agent and/or a therapeutic agent to a subject. Non-limiting examples of dosage regimens include 1 dose daily-24 doses daily, 1 dose weekly-7 doses weekly, 1 dose monthly-30 doses monthly, 1 dose every 2 months-1 dose every year, 1 dose yearly-1 dose every 10 years or more.

The efficacy of a prophylactic agent and/or a therapeutic agent may be assessed by any method. Non-limiting examples of assessing the efficacy of a prophylactic agent and/or a therapeutic agent include monitoring: reduced symptoms associated with the disease, reduction in tumor volume, reduction in metastasis, etc.

Further provided herein, in some embodiments, are methods of monitoring prevention or treatment of disease or disease progression in an immunodeficient model. Prevention or treatment of disease and disease progression may be monitored by any method. Non-limiting examples of monitoring prevention or treatment of disease or disease progression include: measuring temperature, measuring body weight, monitoring movement, and/or assessing sleep/wake cycles.

Provided herein, in some embodiments, are methods of monitoring systemic function of an immunodeficient model having a disease. Systemic function refers to productivity of an organ system in an immunodeficient model with diseased cells and/or tissue. Any organ system in a model (e.g., respiratory, cardiac, digestive, renal, endocrine, or nervous) may be monitored in methods provided herein. Non-limiting examples of monitoring systemic function include: measuring respiratory function (e.g., spirometry, lung capacity and airway resistance, diffusing capacity, blood gas analysis, or cardiopulmonary exercise testing), cardiac function (e.g., cardiac catheterization, pulsed Doppler measures of blood pressure, Doppler blood flow studies, peripheral vessel stiffness and flow velocity), kidney/renal function (proteinuria, creatinine levels, BUN), liver function (albumin, ALT, AST, bilirubin), and neural function (e.g., patch clamp, functional MRI, gait analysis, or balance tests).

In some embodiments, the mouse model provided herein is used to assess cytotoxicity of an agent. Examples of such methods are known and described, for example, in International Application No. WO2018195027A1. As one nonlimiting example, an immunodeficient mouse model of the present disclosure may be engrafted with human HSCs or PBMCs, for example, then administered a therapeutic agent. The blood concentration of various cytokines, such as IFN-γ, IL-2, IL-4, IL-6, IL-10, and/or TNF may be assessed. Determining the level of such cytokines can be indicative of the likelihood that the therapeutic agent will elicit severe cytokine release syndrome in a human subject. Other methods of assessing cytotoxicity are also contemplated herein.

### Nucleic Acids: Engineering and Delivery

The mouse models described herein comprises a nucleic acid encoding human interleukin-3 (IL-3), a nucleic acid encoding human granulocyte/macrophage-colony stimulating factor 2 (GM-CSF), a nucleic acid encoding human stem cell factor (SCF), and a nucleic acid encoding human IL-15. In some embodiments, the mouse models comprise a transgene encoding human IL-3, a transgene encoding human GM-CSF, a transgene encoding human SCF, and a transgene encoding human IL-15, integrated into a mouse genome.

The nucleic acids provided herein, in some embodiments, are engineered. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences).

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene).

A promoter is a nucleotide sequence to which RNA polymerase binds to initial transcription (e.g., ATG). Promoters are typically located directly upstream from (at the 5′ end of) a transcription initiation site. In some embodiments, a promoter is an endogenous promoter. An endogenous promoter is a promoter that naturally occurs in that host animal.

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

An exon is a region of a gene that codes for amino acids. An intron (and other non-coding DNA) is a region of a gene that does not code for amino acids.

A nucleotide sequence encoding a product (e.g., protein), in some embodiments, has a length of 200 base pairs (bp) to 100 kilobases (kb). The nucleotide sequence, in some embodiments, has a length of at least 10 kb. For example, the nucleotide sequence may have a length of at least 15 kb, at least 20 kb, at least 25 kb, at least 30 kb, or at least 35 kb. In some embodiments, the nucleotide sequence has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb.

Any one of the nucleic acids provided herein may have a length of 200 bp to 500 kb, 200 bp to 250 kb, or 200 bp to 100 kb. A nucleic acid, in some embodiments, has a length of at least 10 kb. For example, a nucleic acid may have a length of at least 15 kb, at least 20 kb, at least 25 kb, at least 30 kb, at least 35 kb, at least 50 kb, at least 100 kb, at least 200 kb, at least 300 kb, at least 400 kb, or at least 500 kb. In some embodiments, a nucleic acid has a length of 10 to 500 kb, 20 to 400 kb, 10 to 300 kb, 10 to 200 kb, or 10 to 100 kb. In some embodiments, a nucleic acid has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A nucleic acid may be circular or linear.

The nucleic acids described herein, in some embodiments, include a modification. A modification, with respect to a nucleic acid, is any manipulation of the nucleic acid, relative to the corresponding wild-type nucleic acid (e.g., the naturally-occurring nucleic acid). A genomic modification is thus any manipulation of a nucleic acid in a genome (e.g., in a coding region, non-coding region, and/or regulatory region), relative to the corresponding wild-type nucleic acid (e.g., the naturally-occurring (unmodified) nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g., protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs).

A loss-of-function mutation, as is known in the art, results in a gene product with little or no function. A null mutation, which is a type of loss-of-function mutation, results in a gene product with no function. In some embodiments, an inactivated allele is a null allele. Other examples of loss-of-function mutations includes missense mutations and frameshift mutations.

A nucleic acid, such as an allele or alleles of a gene, may be modified such that it does not produce a detectable level of a functional gene product (e.g., a functional protein). Thus, an inactivated allele is an allele that does not produce a detectable level of a functional gene product (e.g., a functional protein). A detectable level of a protein is any level of protein detected using a standard protein detection assay, such as flow cytometry and/or an ELISA. In some embodiments, an inactivated allele is not transcribed. In some embodiments, an inactivated allele does not encode a functional protein.

Vectors used for delivery of a nucleic acid include minicircles, plasmids, bacterial artificial chromosomes (BACs), and yeast artificial chromosomes. It should be understood, however, that a vector may not be needed. For example, a circularized or linearized nucleic acid may be delivered to an embryo without its vector backbone. Vector backbones are small (˜4 kb), while donor DNA to be circularized can range from >100 bp to 50 kb, for example.

Methods for delivering nucleic acids to mouse embryos for the production of transgenic mice include, but are not limited to, electroporation (see, e.g., Wang W et al. J Genet Genomics 2016; 43(5):319-27; WO 2016/054032; and WO 2017/124086, each of which is incorporated herein by reference), DNA microinjection (see, e.g., Gordon and Ruddle, Science 1981: 214: 1244-124, incorporated herein by reference), embryonic stem cell-mediated gene transfer (see, e.g., Gossler et al., Proc. Natl. Acad. Sci. 1986; 83: 9065-9069, incorporated herein by reference), and retrovirus-mediated gene transfer (see, e.g., Jaenisch, Proc. Natl. Acad. Sci. 1976; 73: 1260-1264, incorporated herein by reference), any of which may be used as provided herein.

### Genomic Editing Methods

Engineered nucleic acids, such as guide RNAs, donor polynucleotides, and other nucleic acid coding sequences, for example, may be introduced to a genome of an embryo or cell (e.g., stem cell) using any suitable method. The present application contemplates the use of a variety of gene editing technologies, for example, to introduce nucleic acids into the genome of an embryo or cell to produce a transgenic rodent. Non-limiting examples include programmable nuclease-based systems, such as clustered regularly interspaced short palindromic repeat (CRISPR) systems, zinc-finger nucleases (ZFNs), and transcription activator-like effector nucleases (TALENs). See, e.g., Carroll D Genetics. 2011; 188(4): 773-782; Joung J K et al. Nat Rev Mol Cell Biol. 2013; 14(1): 49-55; and Gaj T et al. Trends Biotechnol. 2013 July; 31(7): 397-405, each of which is incorporated by reference herein.

In some embodiments, a CRISPR system is used to edit the genome of mouse embryos provided herein. See, e.g., Harms D W et al., Curr Protoc Hum Genet. 2014; 83: 15.7.1-15.7.27; and Inui M et al., Sci Rep. 2014; 4: 5396, each of which are incorporated by reference herein). For example, Cas9 mRNA or protein, one or multiple guide RNAs (gRNAs), and/or a donor nucleic acid can be delivered, e.g., injected or electroporated, directly into mouse embryos at the one-cell (zygote) stage or a later stage to facilitate homology directed repair (HDR), for example, to introduce an engineered nucleic acid (e.g., donor nucleic acid) into the genome.

The CRISPR/Cas system is a naturally occurring defense mechanism in prokaryotes that has been repurposed as an RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the Cas9 endonuclease is from Streptococcus pyogenes (NGG PAM) or Staphylococcus aureus (NNGRRT or NNGRR(N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitidis (NM) Cas9 (NNNNGATT PAM); Streptococcus thermophilus (ST) Cas9 (NNAGAAW PAM); and Treponema denticola (TD) Cas9 (NAAAAC). In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence. See, e.g., Jinek et al., Science, 2012; 337: 816-821 and Deltcheva et al., Nature, 2011; 471: 602-607, each of which is incorporated by reference herein.

In some embodiments, the RNA-guided nuclease and the gRNA are complexed to form a ribonucleoprotein (RNP), prior to delivery to an embryo.

The concentration of RNA-guided nuclease or nucleic acid encoding the RNA-guided nuclease may vary. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl. In some embodiments, the concentration is 100 ng/μl to 500 ng/μl, or 200 ng/μl to 500 ng/μl.

The concentration of gRNA may also vary. In some embodiments, the concentration is 200 ng/μl to 2000 ng/μl. For example, the concentration may be 200, 300, 400, 500, 600, 700, 800, 900, 1000, 1100, 1200, 1300, 1400, 1500, 1600, 1700, 1700, 1900, or 2000 ng/μl. In some embodiments, the concentration is 500 ng/μl to 1000 ng/μl. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl.

In some embodiments, the ratio of concentration of RNA-guided nuclease or nucleic acid encoding the RNA-guided nuclease to the concentration of gRNA is 2:1. In other embodiments, the ratio of concentration of RNA-guided nuclease or nucleic acid encoding the RNA-guided nuclease to the concentration of gRNA is 1:1.

A donor nucleic acid typically includes a sequence of interest flanked by homology arms. Homology arms are regions of the ssDNA that are homologous to regions of genomic DNA located in a genomic locus. One homology arm is located to the left (5′) of a genomic region of interest (into which a sequence of interest is introduced) (the left homology arm) and another homology arm is located to the right (3′) of the genomic region of interest (the right homology arm). These homology arms enable homologous recombination between the ssDNA donor and the genomic locus, resulting in insertion of the sequence of interest into the genomic locus of interest (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded. In some embodiments, the donor DNA is modified, e.g., via phosphorothioation. Other modifications may be made.

### Additional Embodiments

The disclosure, in some aspects, provides an immunodeficient transgenic mouse comprising a transgene encoding human interleuking-3 (IL-3), a transgene encoding human GM-CSF, human granulocyte/macrophage-colony stimulating factor 2 (GM-CSF), a transgene encoding human stem cell factor (SCF), and a transgene encoding human IL-15.

In some embodiments, the transgenic mouse has a non-obese diabetic genetic background. In some embodiments, the transgenic mouse has a severe combined immune deficiency mutation (Prkdc^{scid}). In some embodiments, the transgenic mouse has a null allele of the IL2 receptor common gamma chain (IL2rg^{null}). In some embodiments, the transgenic mouse has a NOD-scid IL2Rgamma^{null} genetic background.

In some embodiments, the transgenic mouse is engrafted with human CD34⁺ hemopoietic stem cells (HSCs). In some embodiments, the transgenic mouse is engrafted with human peripheral blood mononuclear cells (PBMCs).

In some embodiments, peripheral blood of the transgenic mouse comprises human CD45⁺ cells. In some embodiments, the transgenic mouse comprises circulating human CD3⁺ T cells, human CD19⁺ B cells, and human CD33+ myeloid cells.

In some embodiments, the transgenic mouse comprises human NK cells, optionally at levels significantly higher than those in an a humanized NSG-SGM3 control mouse.

In some embodiments, peripheral blood of the transgenic mouse comprises human regulatory T (Treg) cells at levels significantly lower than those in an a humanized NSG-SGM3 control mouse.

In some embodiments, the spleen of the transgenic mouse comprises human PD-1⁺ T cells at levels significantly lower than those in an a humanized NSG-SGM3 control mouse.

In some embodiments, the CD34⁺ human HSCs are engrafted through tail vein injection. In some embodiments, the CD34⁺ human HSCs are engrafted to the mouse at the age of about 4 weeks. In some embodiments, about 5×10⁴ CD34⁺ human HSCs are engrafted to the mouse. In some embodiments, wherein the CD34⁺ human HSCs are engrafted after whole body irradiation of the mouse. In some embodiments, whole body irradiation of the mouse comprises a dose of about 50-150 cGy. In some embodiments, whole body irradiation of the mouse comprises a dose of about 100 cGy.

In some embodiments, the percentage of human CD45⁺ natural killer (NK) cells in peripheral blood of the mouse reaches about 2-10% at about 28 weeks post HSC engraftment. In some embodiments, the percentage of human T regulatory cells in peripheral blood of the mouse reaches about 2-8% at about 16 weeks post-HSC engraftment. In some embodiments, the percentage of T regulatory cells in peripheral blood of the mouse reaches about 5% at about 16 weeks post-HSC engraftment. In some embodiments, PD-1 expression on splenic T cells of the mouse reaches about 15-25% at about 16 weeks post-HSC engraftment. In some embodiments, PD-1 expression on splenic T cells of the mouse reaches about 20% at about 16 weeks post-HSC engraftment.

In some embodiments, the mouse is inoculated with a human patient-derived xenograft (PDX), wherein the HSCs and the PDX are non-HLA matched. In some embodiments, the inoculation comprises subcutaneous or orthotopic implantation. In some embodiments, the human PDX is inoculated to the mouse about 2 weeks after the mouse is engrafted with said CD34⁺ human HSCs. In some embodiments, said human PDX is inoculated to said mouse about 12 weeks after the mouse is engrafted with said CD34⁺ human HSCs. In some embodiments, the human PDX is from a primary patient sample. In some embodiments, the human PDX is from an archived tumor sample that has been passaged as a xenograft for at least one generation. In some embodiments, the human PDX is a xenograft from an ovarian cancer, a lung cancer such as a non-small cell lung cancer (NSCLC), a bladder cancer, a lymphoma (such as AML, CML, ALL, CLL, DLBCL (diffuse large B-cell lymphoma)), a breast cancer such as a triple-negative breast cancer (TNBC), a brain cancer, a pancreatic cancer, a prostate cancer, a colon cancer, a colorectal cancer, an endometrial cancer, a gastric/GIST cancer, a heptocellular cancer, a kidney/renal cancer, a skin cancer (such as melanoma), a soft tissue carcinoma, a sarcoma, or a cancer cell line. In some embodiments, about 5×10⁶ cells of said human PDX are inoculated.

In some embodiments, the exponential growth phase of the tumor occurs at least 40 days after implantation of the tumor. In some embodiments, the tumor volume is less than 600 mm³ for up to 50 days after implantation of the tumor.

In some embodiments, the mouse is administered an anti-cancer compound. In some embodiments, the anti-cancer compound is 5-FU, Avastin, cisplatin, carboplatin, pembrolizumab, docetaxel, or combination thereof.

In some embodiments, the mouse further comprises engrafted human peripheral mononuclear blood cells (PMBCs). In some embodiments, the human PMBCs are engrafted through tail vein injection. In some embodiments, about 1×10⁶ to 5×10⁶ cells of the human PBMCs are engrafted. In some embodiments, about 5×10⁶ cells of the human PBMCs are engrafted. In some embodiments, about 2×10⁶ cells of the human PBMCs are engrafted. In some embodiments, about 1×10⁶ cells of the human PBMCs are engrafted. In some embodiments, the human PBMCs are patient-derived.

In some embodiments, the human PBMCs are engrafted to the mouse at the age of about 4 weeks. In some embodiments, the human PBMCs are engrafted after whole body irradiation of the mouse. In some embodiments, whole body irradiation of the mouse comprises a dose of about 50-200 cGy. In some embodiments, whole body irradiation of the mouse comprises a dose of about 100 cGy. In some embodiments, whole body irradiation of the mouse comprises a dose of about 150-175 cGy.

In some embodiments, the percentage of human CD45⁺ cells in peripheral blood of the mouse reaches about 15-35% at about 8 days post-PBMC engraftment. In some embodiments, the percentage of human CD45⁺ myeloid cells in peripheral blood of the mouse reaches about 0.1-0.2% at about 8 days post-PBMC engraftment. In some embodiments, the percentage of human CD45⁺ NK cells in peripheral blood of the mouse reaches about 5-10% at about 8 days post-PBMC engraftment. In some embodiments, the percentage of activated splenic CD4⁺ T cells of the mouse reaches about 55-70% at about 8 days post-PBMC engraftment. In some embodiments, the percentage of activated splenic CD8⁺ T cells of the mouse reaches about 80-90% at about 8 days post-PBMC engraftment.

In some embodiments, the mouse is engrafted with diseased human cells. In some embodiments, the diseased human cells are selected from blood cells, muscle cells, and neuronal cells. In some embodiments, the diseased human cells are tumor cells. In some embodiments, the tumor cells are primary tumor cells. In some embodiments, the diseased human cells are cancerous cells. In some embodiments, the diseased human cells are non-cancerous cells. In some embodiments, the diseased human cells and the PBMCs are autologous. In some embodiments, the PBMCs and the human immune cells are allogeneic.

In some embodiments, the mouse is further administered a candidate agent for treating cytokine release syndrome (CRS). In some embodiments, a circulating level of a cytokine selected from the group consisting of: interleukin (IL)-6, tumor necrosis factor (TNF), and IL-2 is measured.

Additional embodiments of the present disclosure are encompassed by the following numbered paragraphs:
1. An immunodeficient transgenic mouse comprising a transgene encoding human interleukin-3 (IL-3), a transgene encoding human granulocyte/macrophage-colony stimulating factor 2 (GM-CSF), a transgene encoding human stem cell factor (SCF), and a transgene encoding human IL-15.
2. The transgenic mouse of paragraph 1, wherein the transgenic mouse has a non-obese diabetic genetic background.
3. The transgenic mouse of paragraph 1 or 2, wherein the transgenic mouse has a severe combined immune deficiency mutation (Prkdc^{scid}).
4. The transgenic mouse of any one of the preceding paragraphs, wherein the transgenic mouse has a null allele of the IL2 receptor common gamma chain (IL2rg^{null}).
5. The transgenic mouse of any one of the preceding paragraphs, wherein the transgenic mouse has a NOD-scid IL2Rgamma^{null} genetic background.
6. The transgenic mouse of any one of the preceding paragraphs, wherein the transgenic mouse is engrafted with human CD34⁺ hemopoietic stem cells (HSCs).
7. The transgenic mouse of paragraph 6, wherein peripheral blood of the transgenic mouse comprises human CD45⁺ cells.
8. The transgenic mouse of paragraph 6 or 7, wherein the transgenic mouse comprises circulating human T cells, human B cells, and human myeloid cells.
9. The transgenic mouse of any one of paragraphs 6-8, wherein the transgenic mouse comprises significantly more human NK cells, relative to a humanized NSG-SGM3 control mouse.
10. The transgenic mouse of any one of paragraphs 6-9, wherein the transgenic mouse is engrafted with human xenograft tissue.
11. The transgenic mouse of paragraph 10, wherein the transgenic mouse comprises significantly fewer human regulatory T (Treg) cells in peripheral blood, relative to a humanized NSG-SGM3 control mouse.
12. The transgenic mouse of paragraph 10 or 11, wherein the transgenic mouse comprises significantly fewer human PD-1⁺ T cells, relative to a humanized NSG-SGM3 control mouse.
13. The transgenic mouse of any one of paragraphs 1-5, wherein the transgenic mouse is engrafted with human peripheral blood mononuclear cells (PBMCs).
14. The transgenic mouse of paragraph 13, wherein the transgenic mouse comprises circulating human T cells, human B cells, and human NK cells.
15. The transgenic mouse of paragraph 13 or 14, wherein the transgenic mouse comprises significantly more human CD45⁺ cells in peripheral blood, relative to a humanized NSG-SGM3 control mouse.
16. The transgenic mouse of any one of paragraphs 13-15, wherein the transgenic mouse comprises significantly more human myeloid cells in peripheral blood, relative to a humanized NSG-SGM3 control mouse.
17. The transgenic mouse of any one of paragraphs 13-15, wherein the transgenic mouse comprises significantly more human NK cells in peripheral blood, relative to a humanized NSG-SGM3 control mouse.
18. The transgenic mouse of any one of paragraphs 13-15, wherein the transgenic mouse comprises significantly more activated human T cells in the spleen, relative to a humanized NSG-SGM3 control mouse.
19. A method of producing a transgenic mouse comprising breeding
   (a) an immunodeficient transgenic mouse comprising a transgene encoding human interleukin-3 (IL-3), a transgene encoding human granulocyte/macrophage-colony stimulating factor 2 (GM-CSF), a transgene encoding human stem cell factor (SCF), and
   (b) a transgenic mouse comprising a transgene encoding human IL-15.
20. The method of paragraph 19, wherein the immunodeficient transgenic mouse of (a) has an NSG-SGM3 genetic background.
21. A method comprising administering human xenograft tissue or cells, optionally diseased human xenograft tissue or cells, to the transgenic mouse of any one of paragraphs 1-5.
22. A method comprising administering a dose of human peripheral blood mononuclear cells (PBMCs) to the transgenic mouse of any one of paragraphs 1-5, wherein the dose is less than 1×10⁷ PBMCs.
23. The method of paragraph 22, wherein the dose is about 1×10⁶ to about 5×10⁶ PBMCs.
24. The method of paragraph 23, wherein the dose is about 2×10⁶ PBMCs.
25. The method of paragraph 23, wherein the dose is about 1×10⁶ PBMCs.
26. The method of any one of paragraphs 22-25, wherein the PBMCs are administered intravenously.
27. The method of any one of paragraphs 22-26 further comprising administering to the transgenic mouse an agent that activates human T cells.
28. The method of any one of paragraphs 22-27 further comprising administering to the transgenic mouse a candidate therapeutic agent.
29. The method of paragraph 28, wherein the candidate therapeutic agent is an anti-cancer agent or an immunomodulatory agent.
30. The method of paragraph 29, wherein the immunomodulatory agent is an anti-inflammatory agent, a corticosteroid, or an immunosuppressive agent.
31. The method of any one of paragraphs 28-30, further comprising assaying cytokine levels in the immunodeficient transgenic mouse and/or assaying efficacy of the therapeutic agent.
32. The method of paragraph 31, wherein the cytokine assayed is at least one cytokine selected from the group consisting of IFN-γ, IL-2, IL-4, IL-6, IL-10, and TNF.

Additional aspects, advantages and/or other features of example embodiments of the disclosure will become apparent in view of the following detailed description, taken in conjunction with the accompanying drawings. It should be apparent to those skilled in the art that the described embodiments provided herein are merely exemplary and illustrative and not limiting. Numerous embodiments of modifications thereof are contemplated as falling within the scope of this disclosure and equivalents thereto.

The following examples are provided to further illustrate various non-limiting embodiments and techniques of the present method, including experiments performed in developing the present method. It should be understood, however, that these examples are meant to be illustrative and do not limit the scope of the claims. As would be apparent to skilled artisans, many variations and modifications are intended to be encompassed within the spirit and scope of the disclosure.

### EXAMPLES

### Example 1—NSG-SGM3×IL15 Mouse Humanized with CD34 Hemopoletic Stem Cells (HSCs)

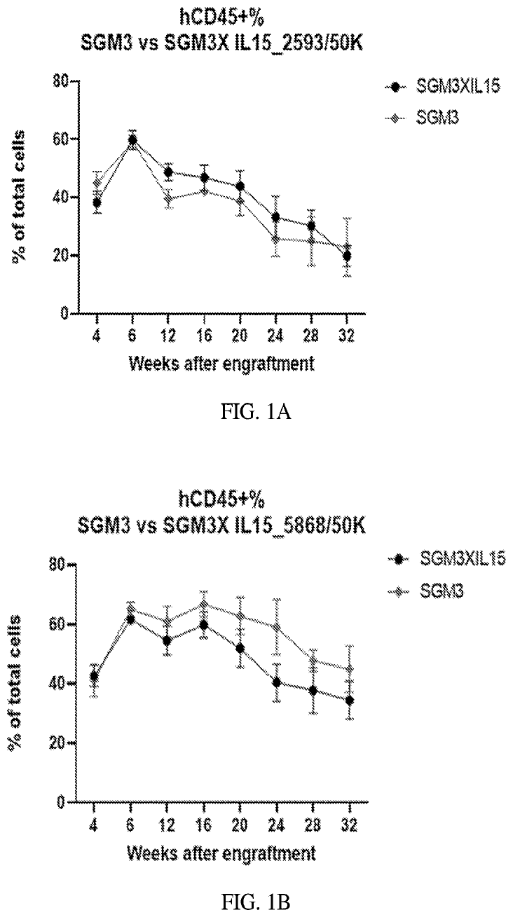

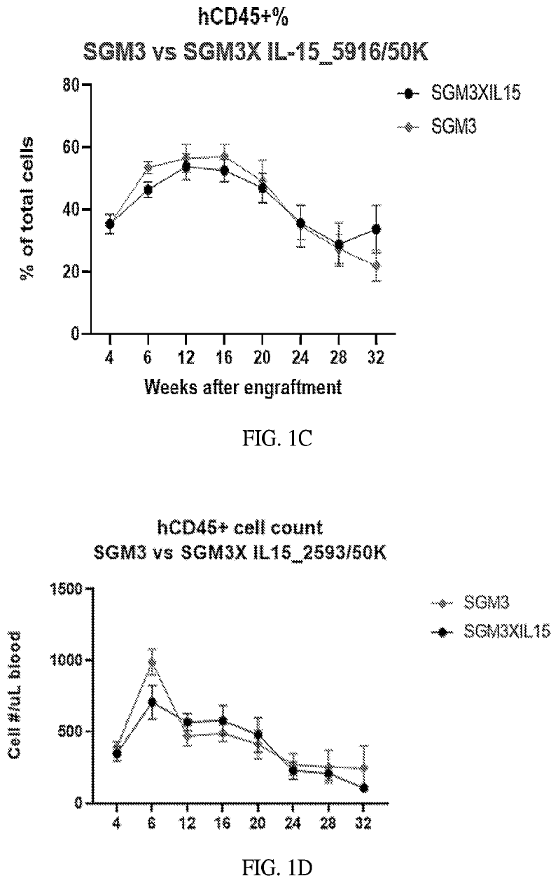

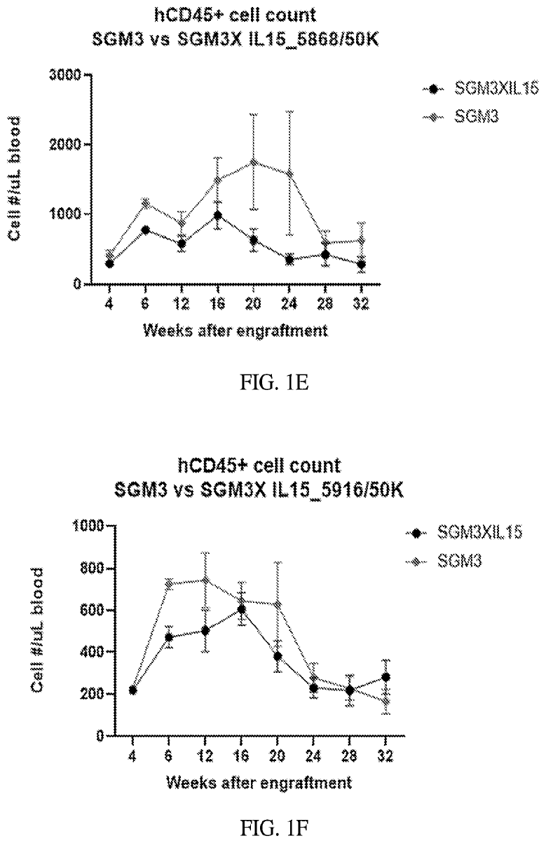

NSG-SGM3 mice and NSG-SGM3×IL15 mice (4 weeks old; N=5/group) were irradiated at 100cGy and injected intravenously with 50,000 CD34+ HSCs derived from umbilical cord blood. Three donors were used. The levels of human CD45+ cells were monitored in the peripheral blood by flow cytometry for up to 32 weeks. Counting beads were added to calculate absolute cell counts. The human CD45+ engraftment time kinetics and engraftment levels are shown in FIGS. 1A-1C (frequency) and FIGS. 1D-1F (cells per microliter of blood). Overall, the kinetics and levels were similar between the two mouse strains.

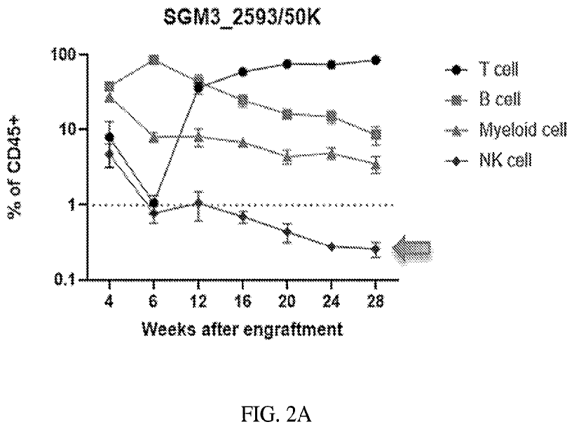

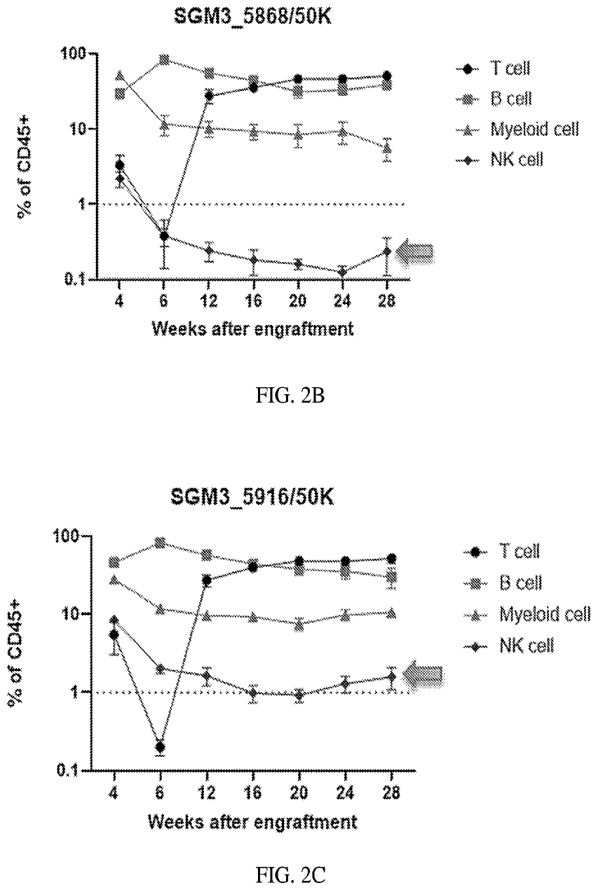

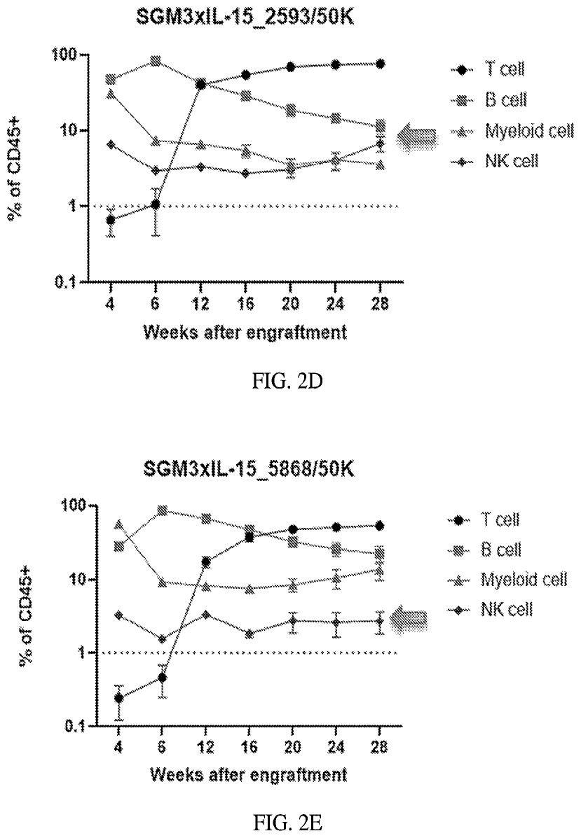

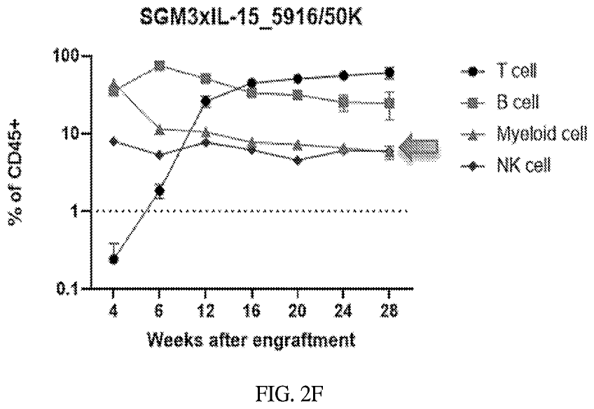

Next, levels of human immune cells were measured in the peripheral blood of NSG-SGM3 mice and NSG-SGM3×IL15 mice at 4, 6, 12, 16, 20, 24, and 28 weeks after engraftment with CD34+ HSCs. The mice underwent the same humanization procedure described above. The results are shown in FIGS. 2A-2C (NSG-SGM3 mice) and FIGS. 2D-2F (NSG-SGM3×IL15 mice). Total NK cell levels (arrows) were higher in the NSG-SGM3×IL15 mice than in the NSG-SGM3 mice and the engraftment levels were well-maintained throughout the 28-week time course for all three donors. Levels of circulating CD3+T, CD19+B, and CD33+myeloid cells were similar between two strains.

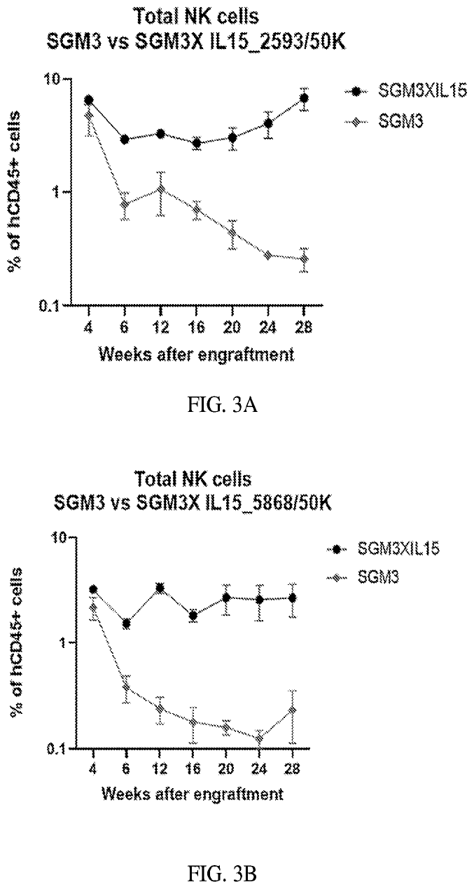

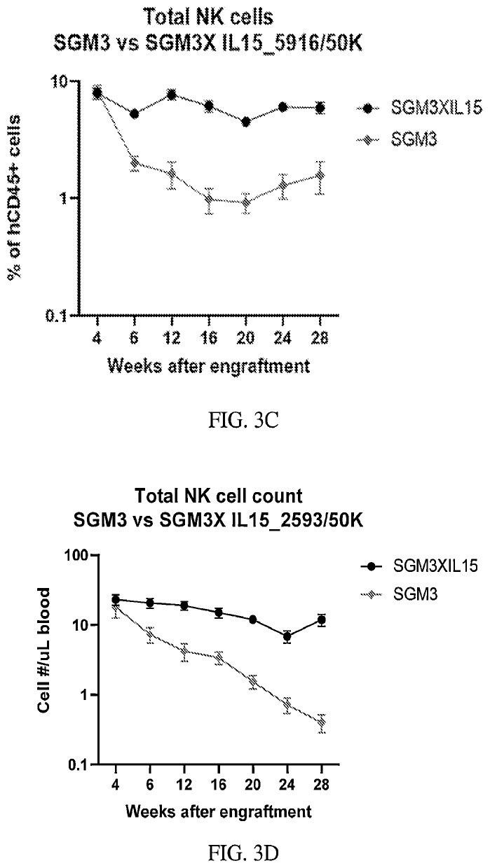

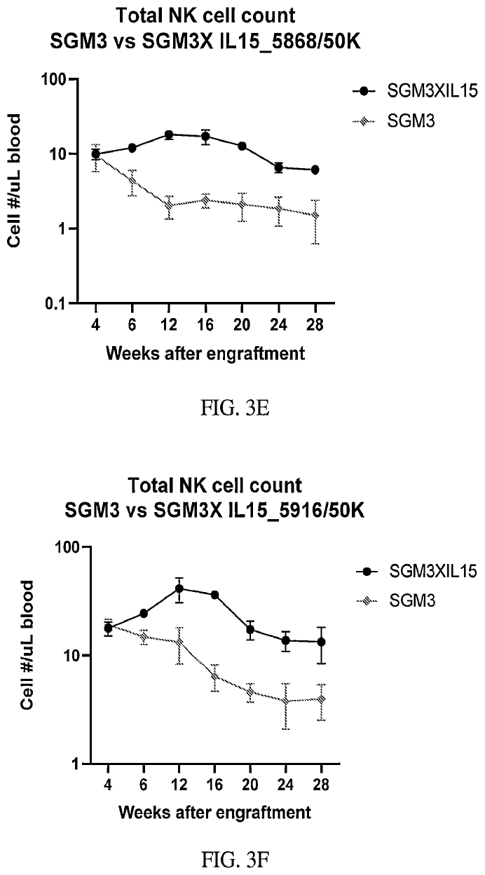

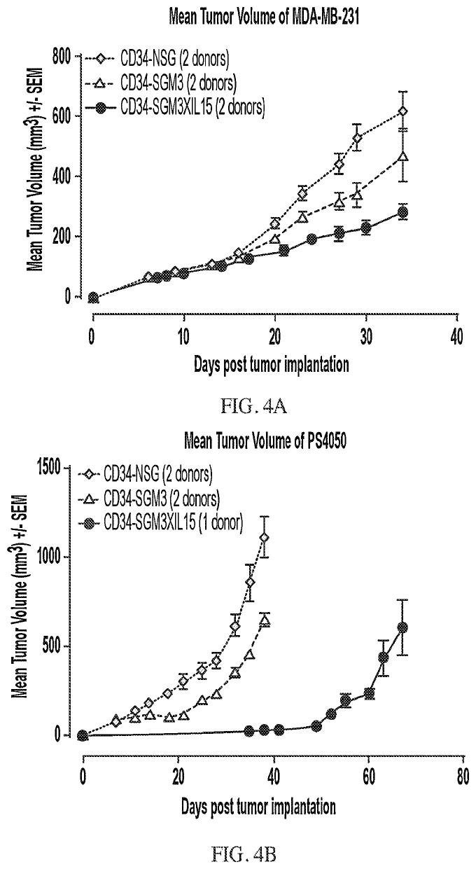

Human NK cell engraftment was further investigated using the same protocol and donors as above. The total NK cell frequency from the three donors is shown in FIGS. 3A-3C and the total NK cells per microliter of blood from the three donors are shown FIGS. 3D-3F. Significantly higher levels of human NK cells were found in the NSG-SGM3×IL15 mice compared to the NSG-SGM3 mice during the 28-week time course. Specifically, the human NK cells frequency was 0.1-1% in the NSG-SGM3 mice and was about 2-10% in the SGM3×IL15 mice.

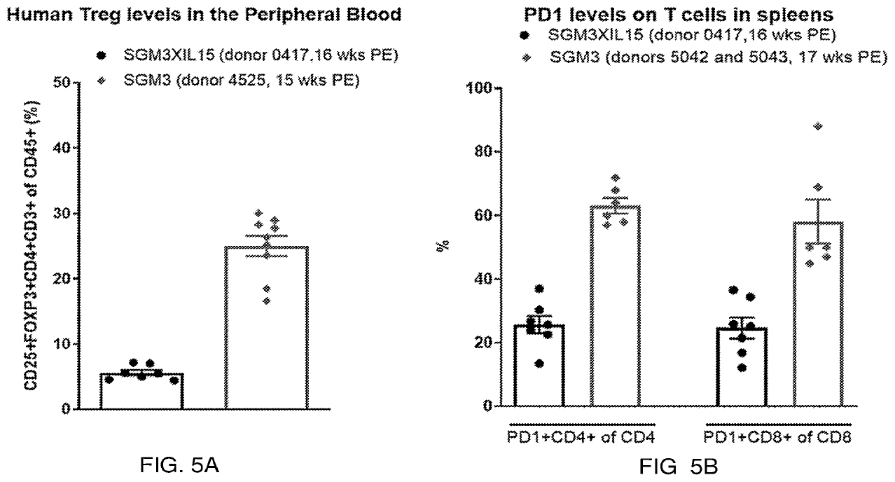

The frequency of regulatory T (Treg) cells in the peripheral blood of blood and PD-1 expression on CD4+T and CD8+T cells in the spleens of NSG-SGM3 mice and NSG-SGM3×IL15 mice engrafted with HSCs as described above was also examined. As shown in FIG. 5A, physiological levels of Treg were observed in the blood of the NSG-SGM3×IL15 mice, while higher levels were observed in the NSG-SGM3 mice (20% vs. 5% of CD45). With respect to PD-1 expression on T cells, FIG. 5B shows that PD-1 expression on CD4T and CD8T cells was higher in the spleen of NSG-SGM3 mice than in NSG-SGM3×IL15 mice (60% vs. 20%).

Therefore, overall, the NSG-SGM3×IL15 mice, humanized with HSCs, demonstrated characteristics closer to the human immune system as compared to the NSG-SGM3 mice.

### Example 2—NSG-SGM3×IL15 Mouse Humanized with CD34 Hemopoletic Stem Cells (HSCs) and Implanted with Patient-Derived Xenografts (PDXs)

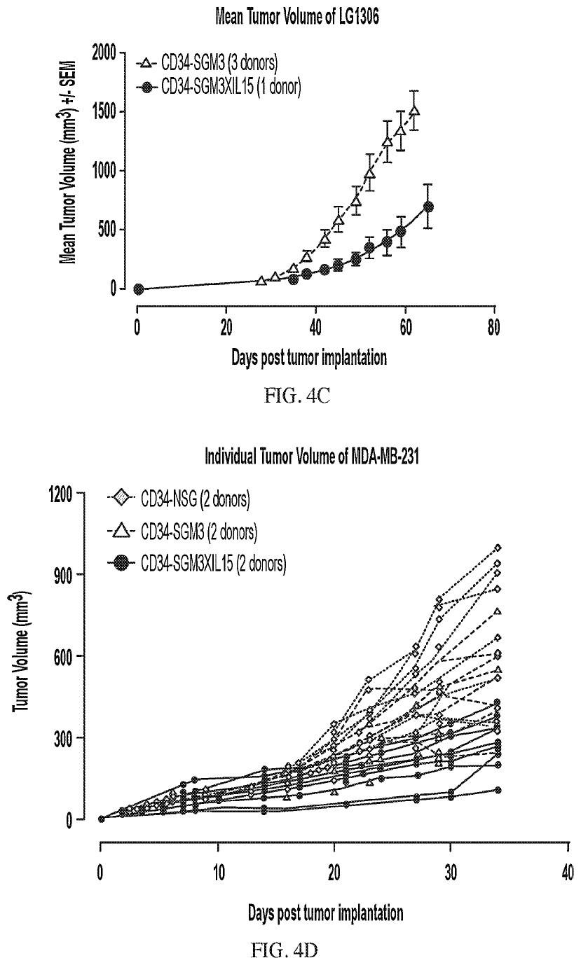

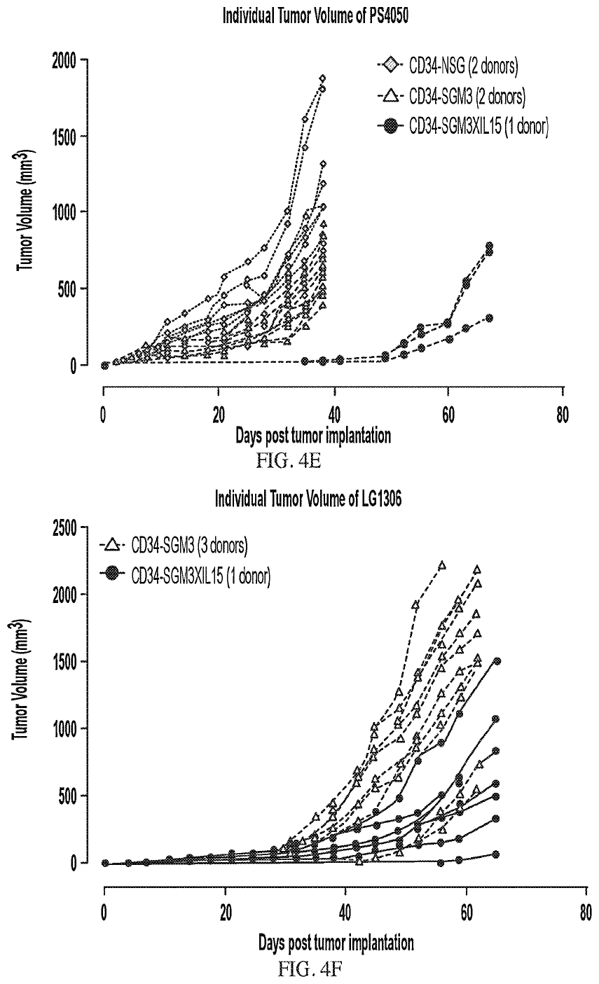

NSG-SGM3×IL15 mice, NSG-SGM3 mice, and NSG mice were humanized with HSCs as described in Example 1. The mice were then subcutaneously implanted with a cell line-derived xenograft (MDA-MB-231) or one of two patient-derived xenografts (PS4050 or LG1306). Each strain of mouse was engrafted with xenografts from one to three donors, as shown in FIGS. 4A-4F. Tumor volume was measured during the time course experiment. FIGS. 4A-4C show the mean tumor volume of the three xenograft models and FIGS. 4E-4F show the individual tumor size of the three models. The growth kinetics of all three xenograft models were significantly delayed in the NSG-SGM3×IL15 mice. This is consistent with a stronger allogenic response against the tumors due to the enhanced human immune system. Moreover, the result agrees with the data of FIG. 5A (Treg frequency) and FIG. 5B (PD-1 expression in T cells), as the NSG-SGM3×IL15 mice appear to have more activated T cells and more abundant NK cells.

### Example 3—NSG-SGM3×IL15 Mouse Humanized with Peripheral Blood Mononuclear Cells (PBMCs)

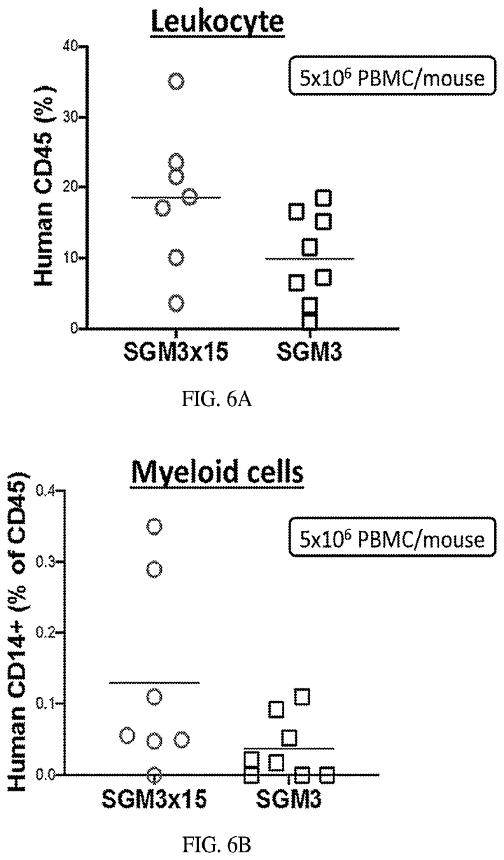

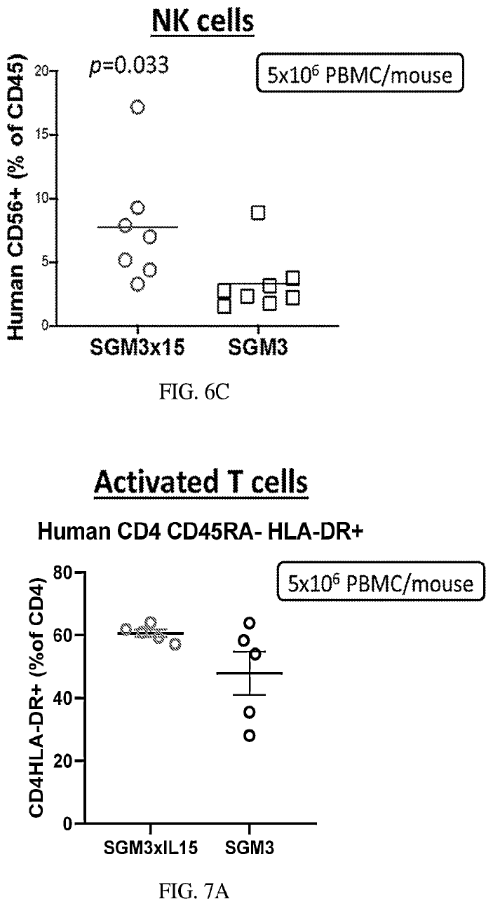

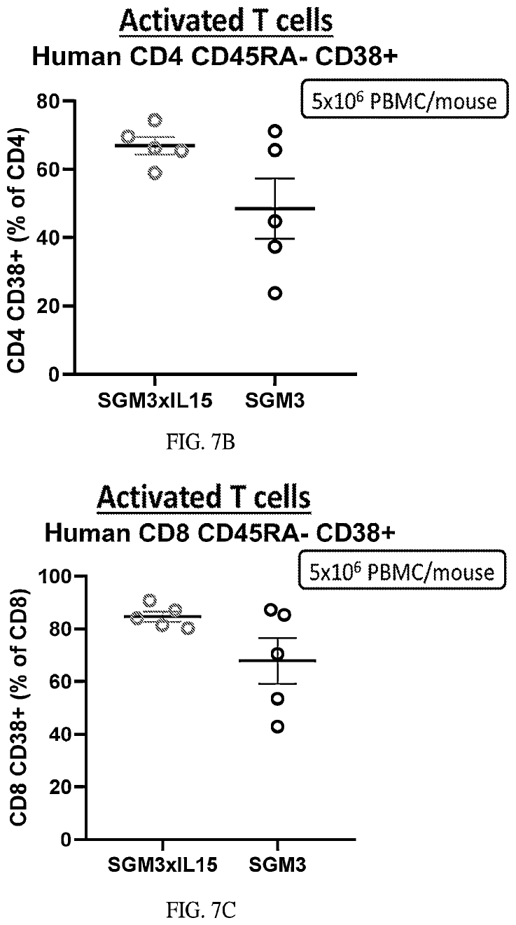

NSG-SGM3×IL15 and NSG-SGM3 mice were humanized with human peripheral blood mononuclear cells (PBMCs) and humanization was confirmed. Briefly, the mice were irradiated (100 cGy) and administered human PBMCs intravenously (5×10⁶/mouse) on day 0. On day 8, blood samples and splenic samples were collected for analysis via flow cytometry. The results are shown in FIGS. 6A-6C (blood) and FIGS. 7A-7C (spleen). The NSG-SGM3×IL15 mice engrafted with 5×10⁶ PBMCs show better humanization (as indicated by higher hCD45+; FIG. 6A) and higher myeloid cell levels (FIG. 6B) compared to the NSG-SGM3 mice. In addition, the NSG-SGM3×IL15 mice showed significantly higher natural killer (NK) cell levels as compared to the NSG-SGM3 mice (FIG. 6C). Taken together, the data demonstrates that the NSG-SGM3×IL15 mice engrafted PBMCs can be a useful model for investigating myeloid and/or NK cells. In the splenic samples, the NSG-SGM3×IL15 mice showed higher activated T cell levels as compared to the NSG-SGM3 mice (FIGS. 7A-7C), which suggests that NSG-SGM3×IL15 can be used as a model for cytokine release syndrome because T cell activation leads to T cell-related cytokine release syndrome.

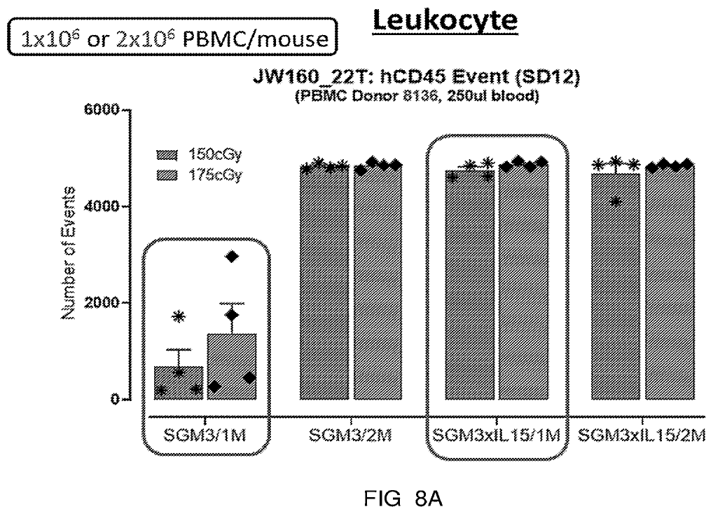

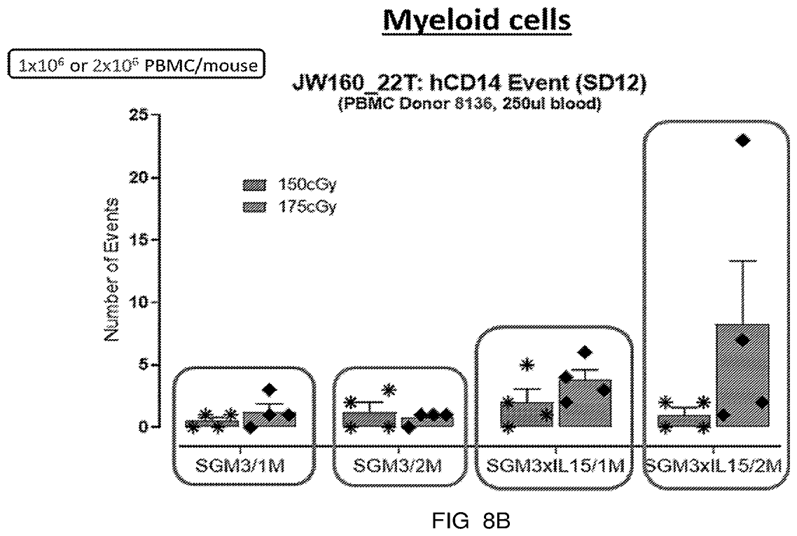

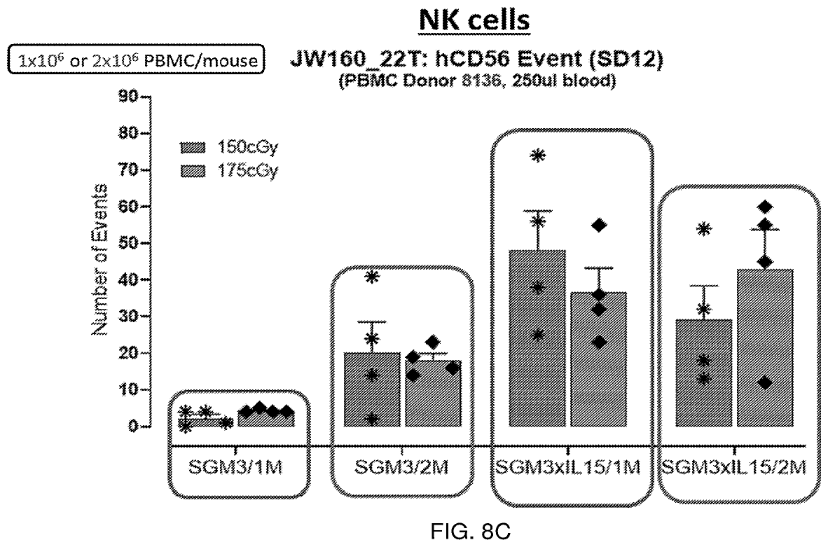

The use of lower levels of PBMCs for humanization was investigated. Briefly, NSG-SGM3×IL15 and NSG-SGM3 mice were irradiated (150 cGy or 175 cGy) and administered human PBMCs intravenously (1×10⁶/mouse or 2×10⁶/mouse) on day 0. On day 12, blood samples were collected for analysis via flow cytometry. The results are shown in FIGS. 8A-8C and demonstrate that the NSG-SGM3×IL15 mice can be humanized with as low as 1×10⁶ PBMC/mouse. This is significantly lower than the quantity of PBMCs typically used (e.g., 10-20×10⁶ PBMCs). Also, with 1×10⁶ or 2×10⁶ PBMC humanization, the NSG-SGM3×IL15 mice showed better humanization (FIG. 8A; human CD45+ cells), higher myeloid cell levels (FIG. 8B), and increased NK cell populations (FIG. 8C) in blood, similar to the pattern seen at the higher PBMC dose (compare to FIGS. 7A-7C).

### Example 4—NSG-SGM3×IL15 Mouse Humanized with Peripheral Blood Mononuclear Cells (PBMCs)—Toxicity Induction

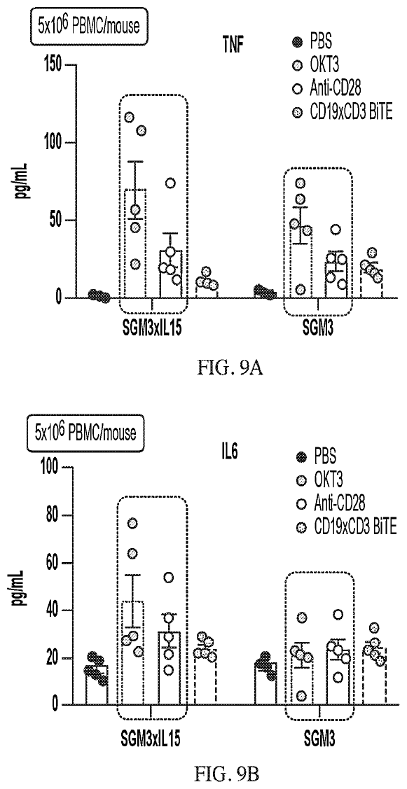

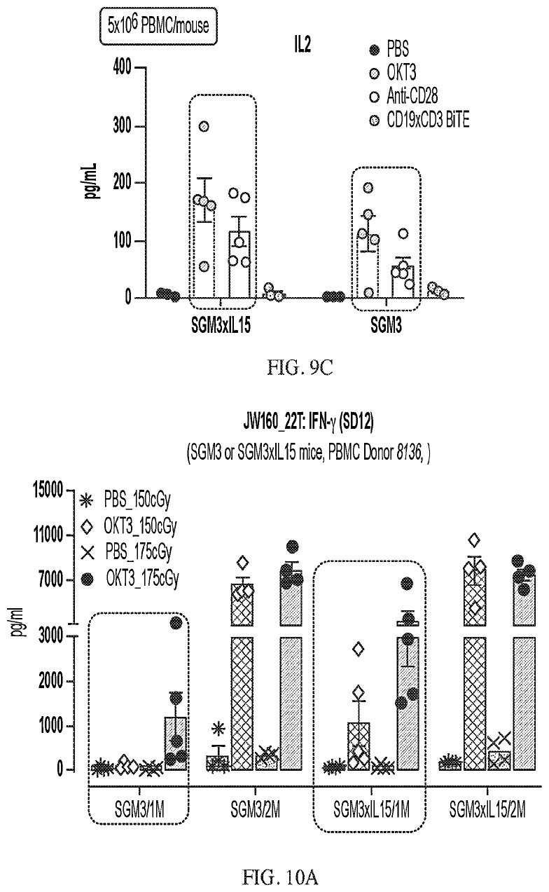

NSG-SGM3×IL15 and NSG-SGM3 mice humanized with human PBMCs and then dosed with various drugs to induce cytokine release syndrome (CRS). Briefly, the mice were irradiated (100 cGy) and administered PBS, OKT3, Anti-CD28, or a CD19×CD3 BiTE. Serum cytokine levels were measured at this point, and the resulting levels of select cytokines are shown in FIGS. 9A-9C. Overall, NSG-SGM3×IL15 mice showed higher cytokine levels when dosed with OKT3 and anti-CD28, which are known to induce cytokine release, in circulation, as compared to NSG-SGM3 mice.

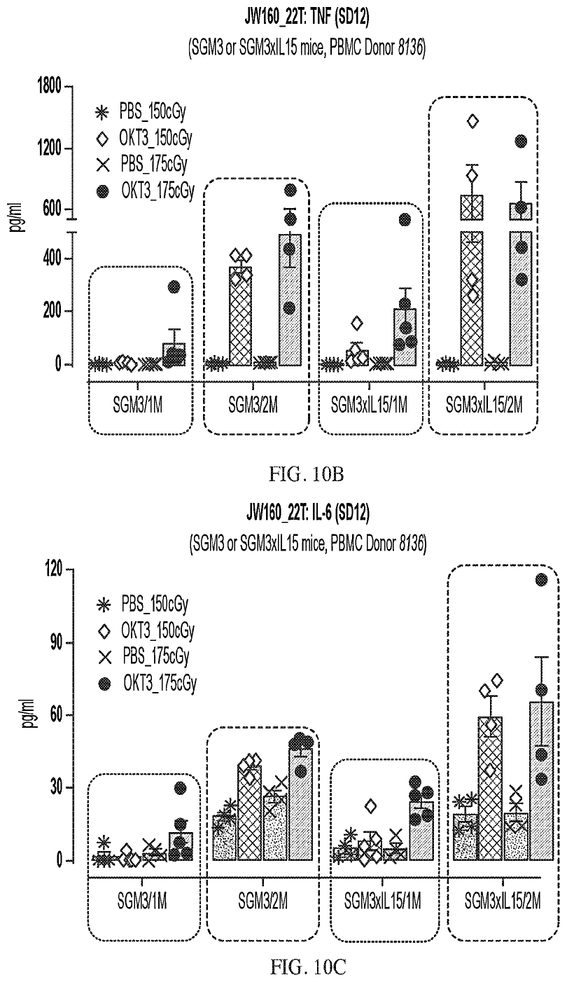

The use of lower levels of PBMCs for humanization was also tested. Briefly, NSG-SGM3×IL15 and NSG-SGM3 mice were irradiated (150 cGy or 175 cGy) and administered human PBMCs intravenously (1×10⁶/mouse or 2×10⁶/mouse) on day 0. On day 12, mice were dosed with PBS or OKT3 and serum cytokine levels were analyzed. The results are shown in FIGS. 10A-10C, and demonstrate that the model still captures human cytokine release with a greater difference between the drug (OKT3) and control (PBS). Even with lower PBMC humanization, the NSG-SGM3×IL15 mice show a tendency of higher cytokine release than the NSG-SGM3 mice, suggesting that the NSG-SGM3×IL15 mice can capture the cytokine release syndrome with significantly fewer number of PBMCs.

### Example 5—Irradiated and Non-Irradiated SGM3×IL15 Mice Humanized with PBMCs

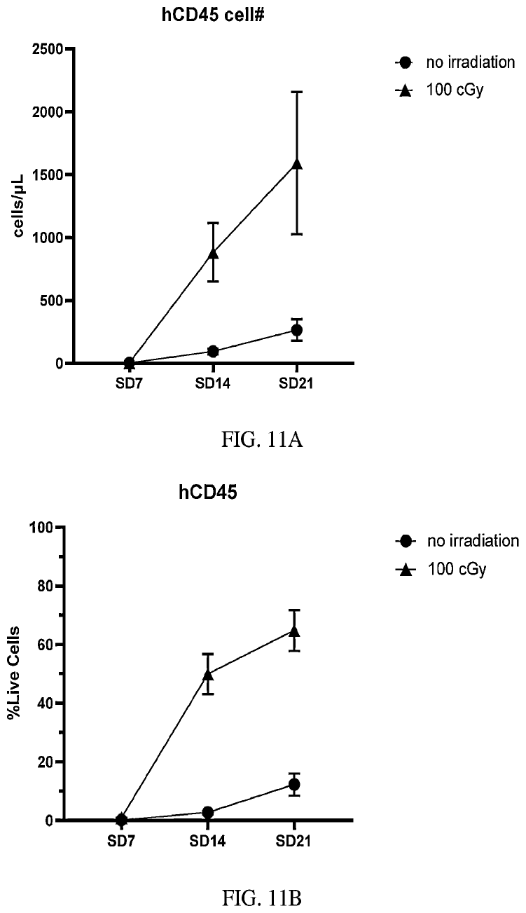

NSG-SGM3×IL15 mice were either irradiated with 100 cGy or not irradiated. Approximately 4 hours later, mice were engrafted with 4 million PBMCs from Donor 3769. Mice were bled once a week and stained using an anti-human CD45 antibody assay. Counting beads were added directly before flow cytometric analysis to determine absolute cell counts. Irradiation led to increased humanization and acceleration of hCD45+ cells in circulation (FIGS. 11A-11B); however, irradiation shortened the lifespan of the mouse. hCD45+ cells in non-irradiated mice expanded until day 49, reaching similar levels observed in irradiated mice at day 21 (data not shown). These results show irradiation enhanced the short term humanization of hCD45 cells, as compared to non-irradiation.

### Example 6—Irradiated and Non-Irradiated SGM3×IL15 Mice Humanized with PBMCs from Two Donors

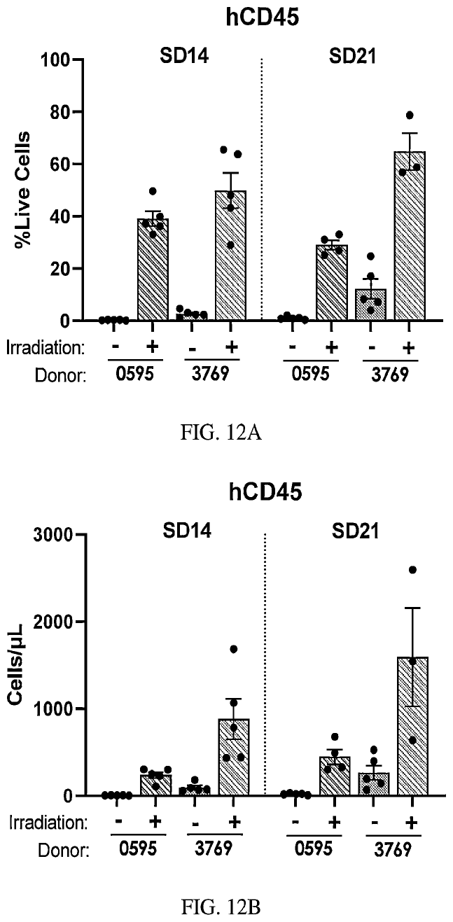

NSG-SGM3×IL15 mice were either irradiated with 100 cGy or not irradiated, and engrafted with 4 million PBMCs from Donors 0595 and 3769. Mice were bled once a week and using an anti-human CD45 antibody assay. Counting beads were added directly before flow cytometric analysis to determine absolute cell counts. As shown in FIGS. 12A-12B, irradiated mice demonstrated enhanced humanization at day 14 and day 21, as compared to non-irradiated mice. Humanization followed a similar pattern in both donors, with some donor variability. In irradiated mice, slightly higher engraftment levels were observed for Donor 3769 than Donor 0595. Donor 3769 also showed higher engraftment numbers than Donor 0595 in non-irradiated mice.

### Example 7—Circulating Human IgG Levels in Irradiated and Non-Irradiated NSG-SGM3×IL15 Mice Humanized with PBMCs from Three Donors

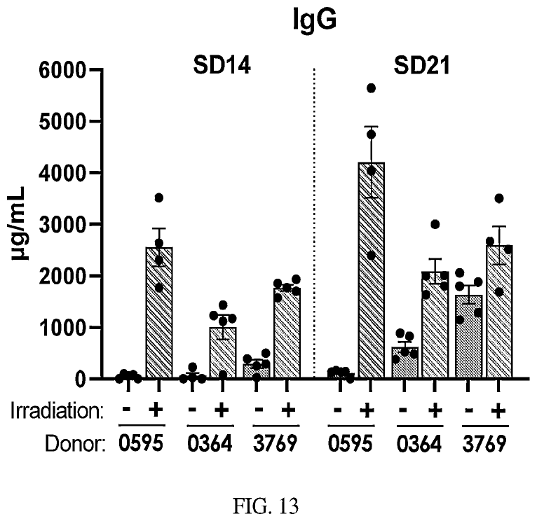

NSG-SGM3×IL15 mice were either irradiated with 100 cGy or not irradiated, and engrafted with 4 million PBMCs from Donors 0364, 0595, and 3769. Mice were bled once a week and analyzed for circulating hIgG levels. All three groups of mice with the donor PBMCs demonstrated IgG levels that increased over time, with irradiated mice producing significantly higher IgG levels on day 14. As shown in FIG. 13, higher IgG levels were observed in irradiated mice at both measured timepoints (day 14, day 21) when compared to non-irradiated mice. Irradiated Donor 0595 mice reached near physiological levels of human IgG as early as day 14 (normal adult IgG level: 600-1600 mg/dL (6000-16000 μg/mL)). Irradiated mice for Donors 0364 and 3769 also showed enhanced IgG production at day 14 when compared to their non-irradiated counterparts. While there was donor variability in the need for irradiation, in all cases, irradiation improved IgG production.

### Example 8—Human Ig Isotypes in Serum for Irradiated and Non-Irradiated NSG-SGM3×IL15 Mice Humanized with PBMCs

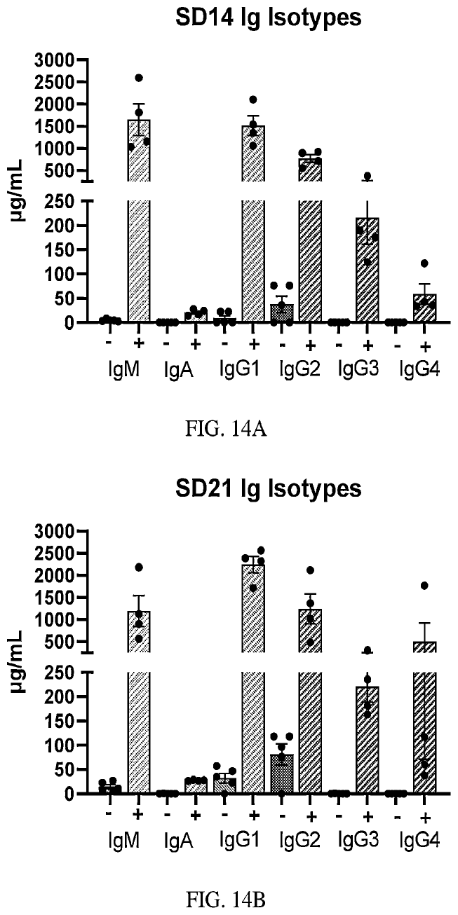

NSG-SGM3×IL15 mice were either irradiated with 100 cGy or not irradiated, engrafted with 4 million PBMCs from Donor 0595, and bled once a week. Serum was analyzed for human IgM, IgA and the four IgG subclasses: IgG1, IgG2, IgG3, IgG4. As shown in FIGS. 14A-14B, irradiation enhanced Ig levels in serum to near physiological levels for IgM, IgG1, IgG2, IgG3 and IgG4 (normal human adult range for: IgG1, 2.80-8.00 mg/ml; IgG2, 1.15-5.70 mg/mL; IgG3, 0.24-1.25 mg/mL; IgG4, 0.052-1.25 mg/mL; IgM,-0.4-2.5 mg/mL). Of the isotypes measured, only one (IgA) did not produce enhanced Ig levels evident in the peripheral blood (normal human adult range for IgA: 0.8-3.0 mg/mL).

### Example 9—Human Immune Cell Population in Irradiated and Non-Irradiated NSG-SGM3×IL15 Mice Humanized with PBMCs

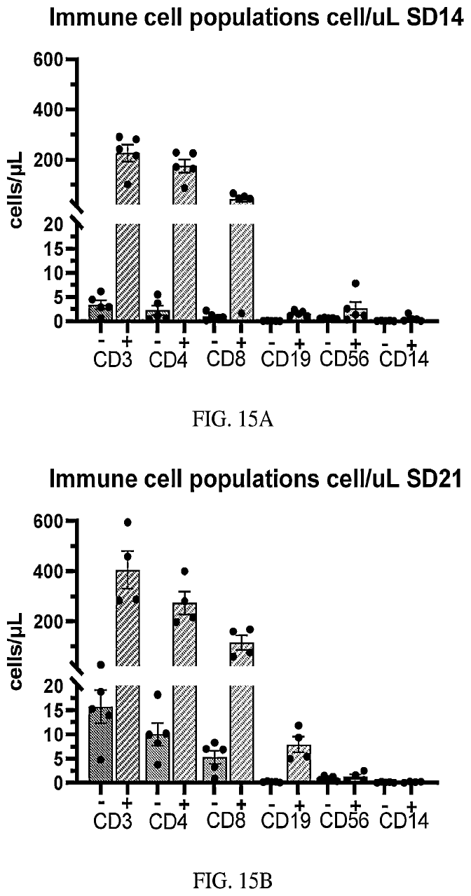

NSG-SGM3×IL15 mice were either irradiated with 100 cGy or not irradiated, and engrafted with 4 million PBMCs from Donor 0595 approximately 4 hours later. Mice were bled once a week and stained using an anti-human CD45, CD3, CD4, CD8, CD19, CD56, CD14 and CD16 antibody assay. Counting beads were added directly before flow cytometric analysis to determine absolute cell counts. As shown in FIGS. 15A-15B, irradiation led to increased hCD45+ cells in circulation as well as expansion of B cells and T cells, while CD56+ and CD14+ numbers remained the same. Unlike CD3+ T cells, where expansion was observed in non-irradiated cells in the peripheral blood, little or no expansion of CD19+ B cells was observed.

### Example 10—Irradiation Dose Influences Humanization and Circulating hIgG Levels in PBMC-Engrafted NSG-SGM3×IL15 Mice

NSG-SGM3×IL15 mice received a dose of irradiation between 0-200 cGy (0 cGy, 50 cGy, 100 cGy, 150 cGy, or 200 cGy) and were engrafted with 4 million PBMCs from Donor 3769. Mice were bled once a week and analyzed for hCD45+ cells and circulating hIgG levels.

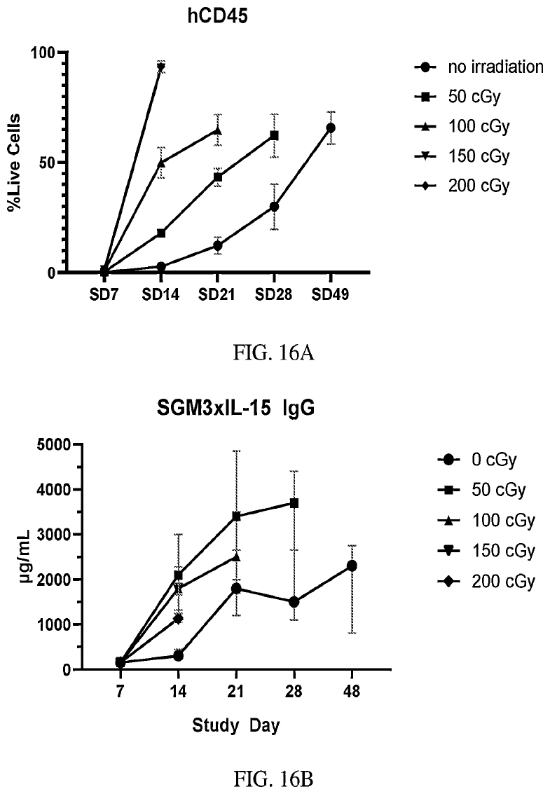

All irradiation doses demonstrated hCD45+ cells and IgG levels that increased over time. As shown in FIGS. 16A-16B, different irradiation doses lead to different outcomes. Irradiation doses of 150 and 200 cGy greatly enhanced humanization at day 14 compared to other conditions but had poorer immunoglobulin production compared to 50 and 100 cGy. 100 cGy had enhanced humanization over 50 cGy at day 14 and 28, but had no improvement of immunoglobulin production, whereas 50 cGy produced the highest immunoglobulin levels of any tested dose. While higher irradiation doses initially resulted in increased hCD45 cells, it also resulted in animals meeting humane euthanasia endpoint criteria before peak hIgG levels could be obtained. Conversely, while non-irradiated mice had longer lifespans and slowed expansion of hCD45, non-irradiated hIgG levels did not reach that of 50-100 cGy groups even though comparable humanization levels were observed. As shown in FIGS. 16A-16B, with irradiation, immunoglobulin levels were enhanced (3500 μg/ml day 28 for 50 cGy; 2500 μg/ml day 21 for 100 cGy) over non-humanized mice (2000 μg/ml at SD 48) that reached the same hCD45+ cell levels over time (SD21 for 100 cGy, SD28 for 50 cGy and SD 48 for 0 cGy.) These results demonstrate that the irradiation enhances immunoglobulin production separately from humanization.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. A method of producing a mouse model of human humoral and cellular immunity, the method comprising:
(a) providing an immunodeficient mouse comprising a transgene encoding human interleukin-3 (IL-3), a transgene encoding human granulocyte/macrophage-colony stimulating factor 2 (GM-CSF), a transgene encoding human stem cell factor (SCF), and a transgene encoding human IL-15;
(b) administering a myeloablative treatment to the immunodeficient mouse; and
(c) administering human peripheral blood mononuclear cells (hPBMCs) to the immunodeficient mouse.

2. The method of claim 1, wherein the myeloablative treatment is irradiation.

3. The method of claim 2, wherein the irradiation is gamma irradiation (cGy).

4. The method of claim 3, wherein the myeloablative treatment is ≤150 cGy.

5. The method of claim 4, wherein the myeloablative treatment is ≤100 cGy.

6. The method of claim 5, wherein the myeloablative treatment is about 50 to about 100 cGy.

7. The method of claim 6, wherein the myeloablative treatment is 40-60 cGy.

8. The method of claim 7, wherein the myeloablative treatment is about 50 cGy.

9. The method of any one of the preceding claims, wherein fewer than 1×10⁷ of the hPBMCs are administered to the immunodeficient mouse.

10. The method of claim 9, wherein 0.5×10⁶-5×10⁶ of the hPBMCs are administered to the immunodeficient mouse.

11. The method of claim 10, wherein about 4×10⁶ of the hPBMCs are administered to the immunodeficient mouse.

12. The method of any one of the preceding claims, wherein the administering of the hPBMCs is within 3 days of the administering of the myeloablative treatment.

13. The method of claim 12, wherein the administering of the hPBMCs is on the same day as the administering of the myeloablative treatment.

14. The method of claim 13, wherein the administering of the hPBMCs is within 6 hours of the administering of the myeloablative treatment.

15. The method of claim 14, wherein the administering of the hPBMCs is at the same time as the administering of the myeloablative treatment.

16. The method of any one of the preceding claims, further comprising administering to the immunodeficient mouse human diseased cells.

17. The method of any one of the preceding claims, further comprising administering to the immunodeficient mouse a human therapeutic or prophylactic agent.

18. The method of claim 17, wherein the human therapeutic agent is selected from human immunomodulatory agents.

19. The method of claim 18, wherein the human immunomodulatory agents are selected from monoclonal antibodies.

20. The method of claim 18, wherein the human immunomodulatory agents are selected from cellular therapies, optionally T cell therapies.

21. The method of any one of the preceding claims, further comprising administering to the immunodeficient mouse a human therapeutic or prophylactic agent is a vaccine.

22. The method of claim 21, wherein the vaccine is a protein antigen or a nucleic acid encoding a protein antigen, optionally a cancer antigen or a pathogenic antigen.

23. The method of any one of claims 17-22, wherein the administering of the candidate human therapeutic agent is within 30 days of administering the myeloablative treatment.

24. The method of claim 23, wherein the administering of the human therapeutic agent is within 28 days of administering the myeloablative treatment.

25. The method of claim 23, wherein the administering of the human therapeutic agent is within 21 days of administering the myeloablative treatment.

26. The method of claim 25, wherein the administering of the human therapeutic agent is within 14 days of administering the myeloablative treatment.

27. The method of any one of the preceding claims, further comprising assaying a sample, optionally a blood sample, from the mouse for one or more human cytokines.

28. The method of claim 24, wherein the one or more human cytokines are selected from interferon gamma (IFN-γ), interleukin (IL)-2, IL-4, IL-6, IL-10, and tumor necrosis factor alpha (TNFα).

29. The method of any one of the preceding claims, further comprising assaying a sample, optionally a blood sample, from the mouse for one or more human immunoglobulins.

30. The method of claim 29, wherein the one or more human immunoglobulins are selected from IgM, IgA, IgG, optionally IgG1, IgG2, IgG3, and IgG4.

31. The method of any one of the preceding claims, further comprising assaying a sample, optionally a blood sample, from the mouse for one or more anti-drug antibodies.

32. A mouse model of human humoral and cellular immunity produced by the method of any one of the preceding claims.

33. An immunodeficient mouse comprising a transgene encoding human interleukin-3 (IL-3), a transgene encoding human granulocyte/macrophage-colony stimulating factor 2 (GM-CSF), a transgene encoding human stem cell factor (SCF), and a transgene encoding human IL-15.

34. The immunodeficient mouse of claim 33, wherein the immunodeficient mouse has a non-obese diabetic genetic background.

35. The immunodeficient mouse of claim 33 or 34, wherein the immunodeficient mouse has a severe combined immune deficiency mutation (Prkdc^{scid}).

36. The immunodeficient mouse of any one of claims 33-35, wherein the immunodeficient mouse has a null allele of the IL2 receptor common gamma chain (IL2rg^{null}).

37. The immunodeficient mouse of any one of claims 33-36, wherein the immunodeficient mouse has a NOD-scid IL2Rgamma^{null} genetic background.

38. The immunodeficient mouse of any one of claims 33-37, wherein the immunodeficient mouse is exposed to myeloablative treatment.

39. The immunodeficient mouse of claim 38, wherein the myeloablative treatment is irradiation.

40. The immunodeficient mouse of any one of claims 33-39, wherein cells of the immunodeficient mouse produce human IgM, IgG1, IgG2, IgG3, and IgG4.

41. The immunodeficient mouse of claim 40, wherein the cells of the immunodeficient mouse produce 6000-16000 μg/mL of human IgG.

42. The immunodeficient mouse of claim 40 or 41, wherein cells of the immunodeficient mouse produce 1000-2000 μl/mL of human IgM.

43. The immunodeficient mouse of any one of claims 39-42, wherein cells of the immunodeficient mouse produce 1500-2500 μl/mL of human IgG1.

44. The immunodeficient mouse of any one of claims 39-43, wherein cells of the immunodeficient mouse produce 500-1500 μl/mL of human IgG2.

45. The immunodeficient mouse of any one of claims 39-44, wherein cells of the immunodeficient mouse produce 150-205 μl/mL of human IgG3.

46. The immunodeficient mouse of any one of claims 39-45, wherein cells of the immunodeficient mouse produce 50-1000 μl/mL of human IgG4.

47. The immunodeficient mouse of any one of claims 32-46, wherein the immunodeficient mouse produces human CD3⁺, CD4⁺, CD8⁺ and CD19⁺ cells.
